(19) United States
(12) Patent Application Publication
(10) Pub. No.: US 2003/0204090 A1
(43) Pub. Date: Oct. 30, 2003

(54) Indolizine compounds

(76) Inventors: Mitsunori Ono, Lexington, MA (US); Teresa Przewloka, Tewksbury, MA (US); David James, Cambridge, MA (US); Dinesh Chimmanamada, (US); Rongzhen Lu, (US); Masazumi Nagai, (US); Keizo Koya, Chestnut Hill, MA (US); Lijun Sun, Harvard, MA (US)

Correspondence Address: FISH & RICHARDSON PC, 225 FRANKLIN ST, BOSTON, MA, 02110, US

(21) Appl. No.: 10/388,332

(22) Filed: Mar. 13, 2003

Related U.S. Application Data
(63) Continuation-in-part of application No. 10/319,401, filed on Dec. 12, 2002.
Application No. 10/319,401 is a continuation-in-part of application No. 10/244,088, filed on Sep. 13, 2002.
(60) Provisional application No. 60/322,020, filed on Sep. 13, 2001.

(51) Int. Cl. C07D 455/02

(52) U.S. Cl. 546/138

(57) ABSTRACT

This invention relates to compounds of Formula (I)

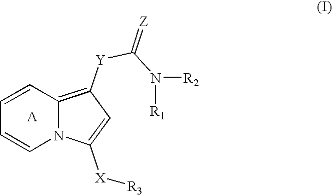

wherein Ring A, X, Y, Z, R₁, R₂ and R₃ are defined herein. These compounds are useful for treating and preventing cancer, inflammatory disorders, autoimmune diseases and other conditions involving PDE4 or elevated levels of cytokines. This invention also relates to pharmaceutical compositions comprising at least one compound of Formula (I) and methods for treating and preventing cancer, inflammatory disorders, autoimmune diseases and other conditions involving PDE4 or elevated levels of cytokines.

### CROSS REFERENCE TO RELATED APPLICATIONS

This application claims the benefit of U.S. Utility application Ser. No. 10/319,401, filed Dec. 12, 2002, which, in turn, claims the benefit of U.S. Utility application Ser. No. 10/244,088, filed Aug. 27, 2002.

### FIELD

This invention relates to biologically active chemical compounds, namely indolizines.

### BACKGROUND

Significant resources have been devoted to research for effective agents against cancer, inflammatory disorders and autoimmune diseases. Despite considerable advances, however, treatments for these conditions are inadequate for a number of reasons.

For example, there are still cancers which simply do not respond or respond poorly to treatments are currently available. Patients with treatable cancers must often undergo chemotherapy with drugs that cause severe side effects. Few of these drugs can be used orally. Perhaps the most serious problem associated with cancer chemotherapy is the development of multi-drug resistance by many tumors. For example, many tumors which initially respond positively to an anti-cancer therapy by decreasing in size or even going into remission often develop resistance to the drug. Tumors that have developed resistance to more than one drug are said to be a “multi-drug resistant”. There is little that can be done to halt or retard further progression of the disease, once a patient's cancer has become multi-drug resistant.

Recent studies have revealed that inhibition of the production or action of tumor necrosis factor alpha (TNFα) has therapeutic effects against inflammatory disorders and autoimmune diseases such as multiple sclerosis, pulmonary fibrosis, atherosclerosis, and Crohn's disease. See Newton et al., J. Med. Chem. (1999) 42(13): 2295-2314. TNFα also plays an important role as a proinflammatory mediator in the development and progression of heart failure. See Mann, D. L., Circ. Res. (2002) 91:988-998. The activity of TNFα can be inhibited by antibodies. However, this immunotherapy can be expensive and inconvenient to treat chronic diseases because the antibodies are administered intravenously once or twice a month in a hospital. Also, antibodies, like most other proteins, tend to be unstable after administration.

Preclinical and clinical studies on phosphodiesterase 4 (PDE4) inhibitors have demonstrated that these agents may find utility in a wide range of inflammatory disorders and autoimmune diseases, including asthma, chronic obstructive pulmonary disease, atopic dermatitis, rheumatoid arthritis, multiple sclerosis, and various neurological disorders. See Doherty, A. M., Current Opinion in Chemical Biology (1999) 3:466-473. No PDE4 inhibitors have been used as drugs to treat inflammatory diseases.

There is therefore still a need for new drugs which overcome one or more of the aforementioned shortcomings of drugs currently used in the treatment of cancer, inflammatory disorders and autoimmune diseases. Desirable properties of new drugs therefore include efficacy against diseases or disorders that are currently untreatable or poorly treatable (e.g., efficacy against multi-drug resistant cancers), oral bioavailability and/or reduced side effects.

### SUMMARY

This invention is based on the discovery that certain indolizine compounds are effective in preventing and treating cancer, inflammatory disorders, autoimmune diseases and other conditions involving PDE4 or elevated levels of cytokines.

This invention features indolizine compounds of Formula (I):

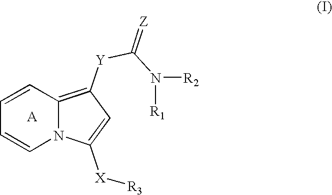

wherein Ring A is substituted or unsubstituted and is optionally fused to an aryl group;

Y is —C(R₄R₅)—, —N(R₄)—, —O—, —S—, —S(O)—, —S(O)₂—, —C(═O)—, —C(═S)—, —C(═O)—N(R₄)—, —C(═N—OR₁₂)—, —C(═N—R₁₂)—, or —N(R₄)—C(═O)—;

Z is ═O, ═S, ═N—OR₁₂ or ═NR₁₂;

R₁ and R₂ are independently —H, an unsubstituted aliphatic group, a substituted aliphatic group, an unsubstituted non-aromatic heterocylic group, a substituted non-aromatic heterocylic group, an unsubstituted aryl group or a substituted aryl group, provided that R₁ and R₂ are not both —H; or alternatively, NR₁R₂, taken together, is a substituted or unsubstituted non-aromatic nitrogen-containing heterocyclic group or a substituted or unsubstituted nitrogen-containing heteroaryl group;

R₃ is a substituted or unsubstituted aryl group or a substituted or unsubstituted aliphatic group;

X is a covalent bond, —C(R₄R₅)—, —N(R₄)—, —O—, —S—, —S(O)—, —S(O)₂—, —C(═O)—, —C(═O)—N(R₄)—, or —N(R₄)—C(═O)—;

each R₄ and R₅ is independently —H or a substituted or unsubstituted aliphatic group;

R₁₂ is —H or a substituted or unsubstituted alkyl group and pharmaceutically acceptable salts and prodrugs thereof.

One embodiment of the present invention relates to pharmaceutical compositions comprising a pharmaceutically acceptable carrier and a compound represented by Formula (I). These pharmaceutical compositions may be used in prophylasis or therapy, for example, to prevent or treat cancer, an inflammatory disorder, an autoimmune disease or other conditions involving PDE4 or elevated levels of cytokines.

Another embodiment of the present invention relates to the use of a compound represented by Formula (I) for the manufacture of a medicament for the prevention or treatment of cancer, an inflammatory disorder, an autoimmune disease or other conditions involving PDE4 or elevated levels of cytokines. The medicament comprises an effective amount of the compound.

Another embodiment of this invention relates to a method of treating a subject with cancer, an inflammatory disorder, an autoimmune disease or other conditions involving PDE4 or elevated levels of cytokines. The method comprises administering to the subject an effective amount of a compound represented by Formula (I) or a pharmaceutical composition comprising a compound represented by Formula (I).

Another embodiment of this invention relates to a method of preventing cancer, an inflammatory disorder, an autoimmune disease and other conditions involving PDE4 or elevated levels of cytokines in a subject susceptible to such disorder, disease or condition. The method comprises administering to the subject an effective amount of a compound represented by Formula (I) or a pharmaceutical composition comprising a compound represented by Formula (I).

Another embodiment of this invention relates to a method of inhibiting TNFα or PDE4 in a cell by contacting the cell with an effective amount of a compound represented by Formula (I) or a pharmaceutical composition comprising a compound represented by Formula (I).

Another embodiment of this invention relates to a method for reducing TNFα levels in a subject comprising administering to the subject an effective amount of a compound represented by Formula (I) or a pharmaceutical composition comprising a compound represented by Formula (I).

Another embodiment of this invention relates to a method for suppressing inflammatory cell activation comprising the step of contacting the cell with an effective amount of a compound represented by Formula (I) or a pharmaceutical composition comprising a compound represented by Formula (I).

The details of various embodiments of the invention are set forth in the description below. Other features, objects, and advantages of the invention will be apparent from the following description and from the claims.

### DETAILED DESCRIPTION

Unless otherwise specified, the below terms (and terms analogous or similar thereto) as used herein are defined as follows:

The term “aryl group” refers to both carbocyclic and heterocyclic aromatic (“heteroaryl”) groups (typically a 5-8 membered monocyclic aromatic ring or a polycyclic aromatic ring or ring system having 5-8 ring members in each ring thereof), such as phenyl, naphthyl, and anthracyl, and heteroaryl groups such as imidazolyl, isoimidazolyl, thienyl, furanyl, pyridyl, pyrimidyl, pyranyl, pyrazolyl, pyrrolyl, pyrazinyl, thiazoyl, thienyl, oxazolyl, isooxazolyl, 1,2,3-trizaolyl, 1,2,4-triazolyl, and tetrazolyl. Aryl groups also include fused polycyclic aromatic ring systems in which a carbocyclic aromatic ring or heteroaryl ring is fused to one or more other carbocyclic aromatic or heteroaryl rings. Examples include benzothienyl, benzofuranyl, indolyl, quinolinyl, benzothiazolyl, benzothienyl, benzooxazolyl, benzoisooxazolyl, benzimidazolyl, quinolinyl, isoquinolinyl and isoindolyl. Nitrogen-containing aryl groups (such as pyridyl) expressly include their N-oxide forms.

The term “aliphatic group” refers to a straight chained, branched or cyclic non-aromatic hydrocarbon which is completely saturated or which contains one or more units of unsaturation. Typically, a straight chained or branched aliphatic group has from 1 to about 10 carbon atoms, preferably from 1 to about 4, and a cyclic aliphatic group has from 3 to about 10 carbon atoms, preferably from 3 to about 8. An aliphatic group is preferably a straight chained or branched alkyl group, e.g, methyl, ethyl, n-propyl, iso-propyl, n-butyl, sec-butyl, tert-butyl, pentyl, hexyl, pentyl or octyl, or a cycloalkyl group with 3 to about 8 carbon atoms. A C₁-C₄ straight chained or branched alkyl group or a C₃-C₈ cyclic alkyl group is also referred to as a “lower alkyl” group and is a preferred subset in the case of each occurrence of an aliphatic group. For the purpose of this invention, “aliphatic group, “alkyl” and terms that incorporate those terms as a prefix or suffix (e.g., alkoxy or aminoalkyl) also includes those moieties where one or more carbons in the group are substituted with oxygen (O), sulfur (S), or nitrogen (N). Further, those groups may optionally be substituted with one or more conventional alkyl substituents, such as amino, alkylamino, alkoxy, alkylthio, oxo, halo, acyl, nitro, hydroxyl, cyano, aryl, alkylaryl, aryloxy, arylthio, arylamino, carbocyclyl, carbocyclyloxy, carbocyclylthio, carbocyclylamino, heterocyclyl, heterocyclyloxy, heterocyclylamino, heterocyclylthio, and the like.

An “alkylene group” is represented by —(CH₂)ₙ—, wherein n is an integer from 1-10, preferably 1-4 and substituted and branched variants thereof.

“Non-aromatic heterocyclic” rings or groups are non-aromatic carbocyclic rings or ring systems which include one or more heteroatoms such as nitrogen, oxygen or sulfur in the ring. Typically, the ring may be five, six, seven or eight-membered or if fused, each ring of the system may have five, six, seven or eight members. Examples include oxazolinyl, thiazolinyl, oxazolidinyl, thiazolidinyl, tetrahydrofuranyl, tetrahyrothiophenyl, morpholino, thiomorpholino, pyrrolidinyl, piperazinyl, piperidinyl, and thiazolidinyl. The terms “heterocyclyl”, “heterocyclic” and the like include both non-aromatic and aromatic heterocycles.

Suitable substituents for Ring A, an aliphatic group, aryl group, or non-aromatic heterocyclic group are those which do not substantially interfere with the prophylactic or therapeutic activity of the disclosed compounds. Examples of suitable substituents include, but are not limited to, —OH, halogen (—Br, —Cl, —I and —F), —OR^{a}, —O—COR^{a}, —COR^{a}, —CN, —NO₂, —COOH, —SO₃H, —NH₂, —NHR^{a}, —N(R^{a}R^{b}), —COOR^{a}, —CHO, —CONH₂, —CONHR^{a}, —CON(R^{a}R^{b}), —NHCOR^{a}, —NRCOR^{a}, —NHCONH₂, —NHCONR^{a}H, —NHCON(R^{a}R^{b}), —NR^{c}CONH₂, —NR^{c}CONR^{a}H, —NR^{c}CON(R^{a}R^{b}), —C(═NH)—NH₂, —C(═NH)—NHR^{a}, —C(═NH)—N(R^{a}R^{b}), —C(═NR^{c})—NH₂, —C(═NR^{c})—NHR^{a}, —C(═NR^{c})—N(R^{a}R^{b}), —NH—C(═NH)—NH₂, —NH—C(═NH)—NHR^{a}, —NH—C(═NH)—N(R^{a}R^{b}), —NH—C(═NR^{c})—NH₂, —NH—C(═NR^{c})—NHR^{a}, —NH—C(═NR^{c)—N(R}^{a}R^{b}), —NR^{d}H—C(═NH)—NH₂, —NR^{d}C(═NH)—NHR^{a}, —NR^{d}C(═NH)—N(R^{a}R^{b}), —NR^{d}—C(═NR^{c})—NH₂, —NR^{d}—C(═NR^{c})—NHR^{a}, —NR^{d}C(═NR^{c})—N(R^{a}R^{b}), —NHNH₂, —NHNHR^{a}, —NHR^{a}R^{b}, —SO₂NH₂, —SO₂NHR^{a}, —SO₂NR^{a}R^{b}, —CH═CHR^{a}, —CH═CR^{a}R^{b}, —CR^{c}═CR^{a}R^{b}, —CR^{c}═CHR^{a}, —CR^{c}═CR^{a}R^{b}, —CCR^{a}, —SH, —SR^{a}, SOₖR^{a} (k is 0, 1 or 2) and —NH—C(═NH)—NH₂. R^{a}-R^{d} are each independently an aliphatic, substituted aliphatic, benzyl, substituted benzyl, aryl or substituted aryl group, preferably an alkyl, benzylic or aryl group. In addition, —NR^{a}R^{d}, taken together, can also form a substituted or unsubstituted non-aromatic heterocyclic group. A non-aromatic heterocyclic group, benzylic group or aryl group can also have an aliphatic or substituted aliphatic group as a substituent. A substituted aliphatic group can also have a non-aromatic heterocyclic ring, a substituted a non-aromatic heterocyclic ring, benzyl, substituted benzyl, aryl or substituted aryl group as a substituent. A substituted aliphatic, non-aromatic heterocyclic group, substituted aryl, or substituted benzyl group may have more than one substituent, which may be the same or different.

Suitable substituents for heteroaryl ring nitrogen atoms having three covalent bonds to other heteroaryl ring atoms include —OH and -alkoxy (preferably C₁-C₄). Substituted heteroaryl ring nitrogen atoms that have three covalent bonds to other heteroaryl ring atoms are positively charged, which is balanced by counteranions such as chloride, bromide, formate, acetate and the like. Examples of other suitable counteranions are provided in the section below directed to suitable pharmacologically acceptable salts.

Suitable substituents for heteroaryl ring nitrogen atoms having two covalent bonds to other heteroaryl ring atoms include alkyl, substituted alkyl (including haloalkyl), phenyl, substituted phenyl, —S(O)₂-(alkyl), —S(O)₂—NH(alkyl) and —S(O)₂—NH(alkyl)₂.

Preferred substituents for Ring A include aryl (e.g., optionally substituted phenyl), halo (e.g., —F, —Cl, and —Br), —C₁-C₄ alkyl, —C₁-C₄ alkoxy, —C₁-C₄ alkoxycarbonyl, —C₁-C₄ haloalkyl, —C₁-C₄ haloalkoxy, —C₁-C₄ haloalkoxycarbonyl, —C₁-C₄ acyl, amido, substituted amido, NO₂, —CN, —OH, —NH₂ and substituted amino. Ring A can have zero, one or more substituents. For substituted amido and substituted amino, the preferred substituent is lower alkyl.

Preferred substitutents for Rings D-T include C₁-C₄ alkyl, C₁-C₄ hydroxyalkyl, N-morpholino, pyrimidyl, C₁-C₄ alkyl substituted with pyrimidyl, —N(C₁-C₄ alkyl)₂, —C(O)NH₂, —C(O)NH(C₁-C₄ alkyl), C(O)N(C₁-C₄ alkyl)₂, —NHC(O)(C₁-C₄ alkyl), —NO₂, C₁-C₄ alkoxy, —C(O)O—CH₂CH₂—N(C₁-C₄ alkyl)₂,

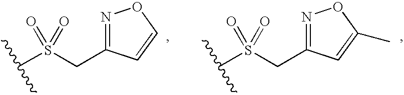

—NH-(phenyl), —NH₂, —CH₂NH—C(O)—O—(C₁-C₄ alkyl), —CH₂NH₂, —Cl, —F, —C(O)—O—(C₁-C₄ alkyl), —C(O)—N—(C₁-C₄ alkyl), C₃-C_{7 -l cycloalkyl, phenyl, —C(O)—N-morpholino, —S—(C}₁-C₄ alkyl), —CN, furyl, —S(O)₂—(C₁-C₄ alkyl), —S(O)₂—NH₂, —S(O)₂—NH(C₁-C₄ alkyl) or —S(O)₂—N(C₁-C₄ alkyl)₂.

The term “cytokine,” as used herein, means any secreted polypeptide that affects the functions of other cells, and that modulates interactions between cells in the immune or inflammatory response. Cytokines include, but are not limited to monokines, lymphokines, and chemokines regardless of which cells produce them. For instance, a monokine is generally referred to as being produced and secreted by a monocyte, however, many other cells produce monokines, such as natural killer cells, fibroblasts, basophils, neutrophils, endothelial cells, brain astrocytes, bone marrow stromal cells, epidermal keratinocytes, and B-lymphocytes. Lymphokines are generally referred to as being produced by lymphocyte cells. Examples of cytokines include, but are not limited to, interleukin-1 (IL-1), interleukin-6 (IL-6), Tumor Necrosis Factor alpha (TNFα), and Tumor Necrosis Factor beta (TNFβ).

The present invention further provides a method of reducing TNFα levels in a subject, comprising the step of administering an effective amount of a compound of Formula (I) to the subject. The term “reducing TNFα levels,” as used herein, means either:

a) decreasing excessive in vivo TNFα levels in a mammal to normal levels or below normal levels by inhibition of the in vivo release of TNFα by all cells, including but not limited to monocytes or macrophages; or

b) inducing a down-regulation, at the translational or transcription level, of excessive in vivo TNFα levels in a mammal to normal levels or below normal levels; or

c) inducing a down-regulation, by inhibition of the direct synthesis of TNFα as a postranslational event.

Moreover, the compounds of the present invention are useful in suppressing inflammatory cell activation. The term “inflammatory cell activation,” as used herein, means the induction by a stimulus (including, but not limited to, cytokines, antigens or auto-antibodies) of a proliferative cellular response, the production of soluble mediators (including but not limited to cytokines, oxygen radicals, enzymes, prostanoids, or vasoactive amines), or cell surface expression of new or increased numbers of mediators (including, but not limited to, major histocompatability antigens or cell adhesion molecules) in inflammatory cells (including but not limited to monocytes, macrophages, T lymphocytes, B lymphocytes, granulocytes, polymorphonuclear leukocytes, mast cells, basophils, eosinophils, dendritic cells, and endothelial cells). It will be appreciated by persons skilled in the art that the activation of one or a combination of these phenotypes in these cells can contribute to the initiation, perpetuation, or exacerbation of an inflammatory condition.

The compounds of this invention may be used to treat or prevent “other conditions involving PDE4 or elevated levels of cytokines”. This term includes, but is not limited to, any disease, condition or disorder which is characterized, mediated or exacerbated by overproduction or activity of TNFα. In addition, this term includes, without limitation, any disease, condition or disorder which is characterized, mediated or exacerbated by overproduction or activity of PDE4 (whether or not it results in elevated level of cytokines). Such conditions include many types of inflammatory disorders, including inflammatory bowel disease (e.g., Crohn's disease), asthma, sepsis, stroke, heart failure, chronic obstructive pulmonary disease, allergic rhinitis, and autoimmune diseases (e.g., arthritis, multiple sclerosis, atherosclerosis, and psoriasis), but will also include other categories of diseases (including, without limitation, cardiomyopathies, such as congestive heart failure, pyrexia, cachexia, cachexia secondary to infection or malignancy, cachexia secondary to acquired immune deficiency syndrome (AIDS), ARC (AIDS-related complex), cerebral malaria, osteoporosis and bone resorption diseases, and fever and myalgias due to infection. In addition, the compounds of the present invention are useful in the treatment of diabetes insipidus and central nervous system disorders, such as depression and multi-infarct dementia).

Also included in the present invention are pharmaceutically acceptable salts of the compounds described herein. Compounds disclosed herein which possess a sufficiently acidic, a sufficiently basic, or both functional groups, and accordingly can react with any of a number of organic or inorganic bases, and inorganic and organic acids, to form a salt. Acids commonly employed to form acid addition salts from compounds with basic groups are inorganic acids such as hydrochloric acid, hydrobromic acid, hydroiodic acid, sulfuric acid, phosphoric acid, and the like, and organic acids such as p-toluenesulfonic acid, methanesulfonic acid, oxalic acid, p-bromophenyl-sulfonic acid, carbonic acid, succinic acid, citric acid, benzoic acid, acetic acid, and the like. Examples of such salts include the sulfate, pyrosulfate, bisulfate, sulfite, bisulfite, phosphate, monohydrogenphosphate, dihydrogenphosphate, metaphosphate, pyrophosphate, chloride, bromide, iodide, acetate, propionate, decanoate, caprylate, acrylate, formate, isobutyrate, caproate, heptanoate, propiolate, oxalate, malonate, succinate, suberate, sebacate, fumarate, maleate, butyne-1,4-dioate, hexyne-1,6-dioate, benzoate, chlorobenzoate, methylbenzoate, dinitrobenzoate, hydroxybenzoate, methoxybenzoate, phthalate, sulfonate, xylenesulfonate, phenylacetate, phenylpropionate, phenylbutyrate, citrate, lactate, gamma-hydroxybutyrate, glycolate, tartrate, methanesulfonate, propanesulfonate, naphthalene-1-sulfonate, naphthalene-2-sulfonate, mandelate, and the like.

Base addition salts include those derived from inorganic bases, such as ammonium or alkali or alkaline earth metal hydroxides, carbonates, bicarbonates, and the like. Such bases useful in preparing the salts of this invention thus include sodium hydroxide, potassium hydroxide, ammonium hydroxide, potassium carbonate, and the like.

Prodrugs of the compounds of this invention are also contemplated herein. The term “prodrug” means a derivative of a compound that can hydrolyze, oxidize, or otherwise react under biological conditions (in vitro or in vivo) to provide a compound of this invention. Prodrugs may only become active upon such reaction under biological conditions, but they may have activity in their unreacted forms. Examples of prodrugs contemplated in this invention include, but are not limited to, analogs or derivatives of compounds of Formula (I) that comprise biohydrolyzable moieties such as biohydrolyzable amides, biohydrolyzable esters, biohydrolyzable carbamates, biohydrolyzable carbonates, biohydrolyzable ureides, and biohydrolyzable phosphate analogues. Other examples of prodrugs include derivatives of compounds of Formula (I) that comprise —NO, —NO₂, —ONO, or —ONO₂ moieties. Prodrugs can typically be prepared using well-known methods, such as those described by 1 Burger's Medicinal Chemistry and Drug Discovery (1995) 172-178, 949-982 (Manfred E. Wolff ed., 5^{th} ed).

As used herein and unless otherwise indicated, the terms “biohydrolyzable amide”, “biohydrolyzable ester”, “biohydrolyzable carbamate”, “biohydrolyzable carbonate”, “biohydrolyzable ureide” and “biohydrolyzable phosphate analogue” mean an amide, ester, carbamate, carbonate, ureide, or phosphate analogue, respectively, that either: 1) does not destroy the biological activity of the compound and confers upon that compound advantageous properties in vivo, such as uptake, duration of action, or onset of action; or 2) is itself biologically inactive but is converted in vivo to a biologically active compound. Examples of biohydrolyzable amides include, but are not limited to, lower alkyl amides, α-amino acid amides, alkoxyacyl amides, and alkylaminoalkylcarbonyl amides. Examples of biohydrolyzable esters include, but are not limited to, lower alkyl esters, alkoxyacyloxy esters, alkyl acylamino alkyl esters, and choline esters. Examples of biohydrolyzable carbamates include, but are not limited to, lower alkylamines, substituted ethylenediamines, amino acids, hydroxyalkylamines, heterocyclic and heteroaromatic amines, and polyether amines.

Certain compounds of the invention may contain one or more chiral centers and/or double bonds and, therefore, exist as stereoisomers, such as double-bond isomers (i.e., geometric isomers), enantiomers, or diastereomers. According to the invention, the chemical structures depicted herein, and therefore the compounds of the invention, encompass all of the corresponding compounds' enantiomers and stereoisomers, that is, both the stereomerically pure form (e.g., geometrically pure, enantiomerically pure, or diastereomerically pure) and enantiomeric and stereoisomeric mixtures.

As used herein, a racemic mixture means about 50% of one enantiomer and about 50% of is corresponding enantiomer relative to all chiral centers in the molecule. The invention encompasses all enantiomerically-pure, enantiomerically-enriched, diastercomerically pure, diastereomerically enriched, and racemic mixtures of the compounds of Formula (I).

Enantiomeric and diastereomeric mixtures can be resolved into their component enantiomers or stereoisomers by well known methods, such as chiral-phase gas chromatography, chiral-phase high performance liquid chromatography, crystallizing the compound as a chiral salt complex, or crystallizing the compound in a chiral solvent. Enantiomers and diastereomers can also be obtained from diastereomerically- or enantiomerically-pure intermediates, reagents, and catalysts by well known asymmetric synthetic methods.

Without wishing to be bound by theory, the compounds of this invention inhibit TNFα and/or PDE4. In this context “inhibit” refers to interfering with the production or activity of TNFα or PDE4 in a direct or an indirect fashion. For example, the compounds of this invention may block production of TNFα by interfering at the transcriptional, translational or post-translational level or block activity of the PDE4 enzyme. In some cases, compounds of this invention will inhibit TNFα but not PDE4. The ability of a compound of this invention to inhibit TNFα or PDE4 may be readily evaluated using the techniques described herein and other techniques known to those of skill in the art.

The compounds of this invention can be used to prevent or treat TNFα- or PDE4-related diseases other than inflammatory disorders, autoimmune diseases and cancer. Further, it should be noted that these compounds can bring about their therapeutic and prophylactic effects either via inhibition of TNFα or PDE4, or via another unrelated mechanism. These compounds also have utility in screening, research, and diagnosis.

The compounds of this invention can be used to treat subjects with cancer, including multi-drug resistant cancers. A cancer is resistant to a drug when it resumes a normal rate of tumor growth while undergoing treatment with the drug after the tumor had initially responded to the drug. A tumor “responds to a drug” when it exhibits a decrease in tumor mass or a decrease in the rate of tumor growth. The term “multi-drug resistant cancer” refers to cancer that is resistant to two or more drugs, typically five or more.

As used herein, the term “cancer” means a disease, condition or disorder characterized by a proliferation of cells with loss of normal controls resulting in unregulated growth, lack of differentiation, local tissue invasion, and metastasis. Cancer is characterized primarily by an increase in the number of abnormal cells derived from a given normal tissue, invasion of adjacent tissues by these abnormal cells, and lymphatic or blood-borne spread of malignant cells to regional lymph nodes and to distant sites (metastasis). Clinical data and molecular biologic studies indicate that cancer is a multistep process that begins with minor preneoplastic changes, which may under certain conditions progress to neoplasia. Pre-malignant abnormal cell growth is exemplified by hyperplasia, metaplasia, or most particularly, dysplasia (for review of such abnormal growth conditions, see Robbins and Angell (1976) Basic Pathology, 2d Ed., W. B. Saunders Co., Philadelphia, 68-79.) The compounds of this invention may be used to prevent or treat cancer in each of these cases and the term “cancer” as used herein encompasses all such abnormal growth conditions whether they are considered cancerous or pre-cancerous.

Hyperplasia is a form of controlled cell proliferation involving an increase in cell number in a tissue or organ, without significant alteration in structure or function. As but one example, endometrial hyperplasia often precedes endometrial cancer. Metaplasia is a form of controlled cell growth in which one type of adult or fully differentiated cell substitutes for another type of adult cell. Metaplasia can occur in epithelial or connective tissue cells. Atypical metaplasia involves a somewhat disorderly metaplastic epithelium. Dysplasia is frequently a forerunner of cancer, and is found mainly in the epithelia; it is the most disorderly form of non-neoplastic cell growth, involving a loss in individual cell uniformity and in the architectural orientation of cells. Dysplastic cells often have abnormally large, deeply stained nuclei, and exhibit pleomorphism. Dysplasia characteristically occurs where there exists chronic irritation or inflammation, and is often found in the cervix, respiratory passages, oral cavity, and gall bladder. The neoplastic lesion may evolve clonally and develop an increasing capacity for invasion, growth, metastasis, and heterogeneity, especially under conditions in which the neoplastic cells escape the host's immune surveillance (Roitt, I., Brostoff, J and Kale, D. (1993) Immunology, 3rd ed., Mosby, St. Louis, 17.1-17.12).

Cancers that can be treated or prevented by the compounds and methods of the present invention include, but are not limited to human sarcomas and carcinomas, e.g., fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, Iciomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, testicular tumor, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, neuroblastoma, retinoblastoma; leukemias, e.g., acute lymphocytic leukemia and acute myelocytic leukemia (myeloblastic, promyelocytic, myclomonocytic, monocytic and erythroleukemia); chronic leukemia (chronic myelocytic (granulocytic) leukemia and chronic lymphocytic leukemia); and polycythemia vera, lymphoma (Hodgkin's disease and non-Hodgkin's disease), multiple myeloma, Waldenstrom's macroglobulinemia, and heavy chain disease. In the case of cancer, the term “treating” includes achieving, partially or substantially, one or more of the following: arresting the growth or spread of a cancer, reducing the extent of a cancer (e.g., reducing size of a tumor or reducing the number of affected sites), inhibiting the growth rate of a cancer, and ameliorating or improving a clinical symptom or indicator associated with a cancer (such as tissue or serum components).

As used herein, the term “asthma” means a pulmonary disease, disorder or condition characterized by reversible airway obstruction, airway inflammation, and increased airway responsiveness to a variety of stimuli.

The compounds of this invention can be used to treat subjects with autoimmune diseases. As used herein, the term “autoimmune disease” means a disease, disorder or condition caused by the immune system of an animal mistakenly attacking itself, thereby targeting the cells, tissues, and/or organs of the animal's own body. For example, the autoimmune reaction is directed against the brain in multiple sclerosis and the gut in Crohn's disease. In other autoimmune diseases such as systemic lupus erythematosus (lupus), affected tissues and organs may vary among individuals with the same disease. One person with lupus may have affected skin and joints whereas another may have affected skin, kidney, and lungs. Ultimately, damage to certain tissues by the immune system may be permanent, as with destruction of insulin-producing cells of the pancreas in Type 1 diabetes mellitus. Specific autoimmune diseases that may be ameliorated using the compounds and methods of this invention include without limitation, autoimmune diseases of the nervous system (e.g., multiple sclerosis, myasthenia gravis, autoimmune neuropathies such as Guillain-Barré, and autoimmune uveitis), autoimmune diseases of the blood (e.g., autoimmune hemolytic anemia, pernicious anemia, and autoimmune thrombocytopenia), autoimmune diseases of the blood vessels (e.g., temporal arteritis, anti-phospholipid syndrome, vasculitides such as Wegener's granulomatosis, and Behcet's disease), autoimmune diseases of the skin (e.g., psoriasis, dermatitis herpetiformis, pemphigus vulgaris, and vitiligo), autoimmune diseases of the gastrointestinal system (e.g., Crohn's disease, ulcerative colitis, primary biliary cirrhosis, and autoimmune hepatitis), autoimmune diseases of the endocrine glands (e.g., Type 1 or immune-mediated diabetes mellitus, Grave's disease. Hashimoto's thyroiditis, autoimmune oophoritis and orchitis, and autoimmune disease of the adrenal gland); and autoimmune diseases of multiple organs (including connective tissue and musculoskeletal system diseases) (e.g., rheumatoid arthritis, systemic lupus erythematosus, scleroderma, polymyositis, dermatomyositis, spondyloarthropathies such as ankylosing spondylitis, and Sjogren's syndrome). In addition, other immune system mediated diseases, such as graft-versus-host disease and allergic disorders, are also included in the definition of autoimmune diseases herein. Because a number of autoimmune disorders are caused by inflammation, there is some overlap between disorders that are considered autoimmune diseases and inflammatory disorders. For the purpose of this invention, in the case of such an overlapping disorder, it may be considered either an autoimmune disease or an inflammatory disorder. “Treatment of an autoimmune disease” herein refers to administering a composition of the invention to a subject, who has an autoimmune disease, a symptom of such a disease or a predisposition towards such a disease, with the purpose to cure, relieve, alter, affect, or prevent the autoimmune disease, the symptom of it, or the predisposition towards it.

As used herein, the term “allergic disorder” means a disease, condition or disorder associated with an allergic response against normally innocuous substances. These substances may be found in the environment (such as indoor air pollutants and aeroallergens) or they may be non-environmental (such as those causing dermatological or food allergies). Allergens can enter the body through a number of routes, including by inhalation, ingestion, contact with the skin or injection (including by insect sting). Many allergic disorders are linked to atopy, a predisposition to generate the allergic antibody IgE. Because IgE is able to sensitize mast cells anywhere in the body, atopic individuals often express disease in more than one organ. For the purpose of this invention, allergic disorders include any hypersensitivity that occurs upon re-exposure to the sensitizing allergen, which in turn causes the release of inflammatory mediators. Allergic disorders include without limitation, allergic rhinitis (e.g., hay fever), sinusitis, rhinosinusitis, chronic or recurrent otitis media, drug reactions, insect sting reactions, latex reactions, conjunctivitis, urticaria, anaphylaxis and anaphylactoid reactions, atopic dermatitis, asthma and food allergies.

The compounds of this invention can be used to prevent or to treat subjects with inflammatory disorders. As used herein, an “inflammatory disorders” means a disease, disorder or condition characterized by inflammation of the body tissue. These include local inflammatory responses and systemic inflammation. Examples of such inflammatory disorders include: transplant rejection; chronic inflammatory disorders of the joints, including arthritis, rheumatoid arthritis, osteoarthritis and bone diseases associated with increased bone resorption; inflammatory bowel diseases such as ileitis, ulcerative colitis, Barrett's syndrome, and Crohn's disease; inflammatory lung disorders such as asthma, adult respiratory distress syndrome, and chronic obstructive airway disease; inflammatory disorders of the eye including corneal dystrophy, trachoma, onchocerciasis, uveitis, sympathetic ophthalmitis and endophthalmitis; chronic inflammatory disorders of the gums, including gingivitis and periodontitis; tuberculosis; leprosy; inflammatory diseases of the kidney including uremic complications, glomerulonephritis and nephrosis; inflammatory disorders of the skin including sclerodermatitis, psoriasis and eczema; inflammatory diseases of the central nervous system, including chronic demyelinating diseases of the nervous system, multiple sclerosis, AIDS-related neurodegeneration and Alzheimer's disease, infectious meningitis, encephalomyelitis, Parkinson's disease, Huntington's disease, amyotrophic lateral sclerosis and viral or autoimmune encephalitis; autoimmune diseases, immune-complex vasculitis, systemic lupus and erythematodes; systemic lupus erythematosus (SLE); and inflammatory diseases of the heart such as cardiomyopathy, ischemic heart disease hypercholesterolemia, atherosclerosis); as well as various other diseases with significant inflammatory components, including preeclampsia; chronic liver failure, brain and spinal cord trauma, cancer). There may also be a systemic inflammation of the body, exemplified by gram-positive or gram negative shock, hemorrhagic or anaphylactic shock, or shock induced by cancer chemotherapy in response to pro-inflammatory cytokines, e.g., shock associated with pro-inflammatory cytokines. Such shock can be induced, e.g., by a chemotherapeutic agent used in cancer chemotherapy. “Treatment of an inflammatory disorder” herein refers to administering a composition of the invention to a subject, who has an inflammatory disorder, a symptom of such a disorder or a predisposition towards such a disorder, with the purpose to cure, relieve, alter, affect, or prevent the inflammatory disorder, the symptom of it, or the predisposition towards it.

An “effective amount” is the quantity of compound in which a beneficial outcome is achieved when the compound is administered to a subject or alternatively, the quantity of compound that possess a desired activity in-vivo or in-vitro. In the case of cancer, a beneficial clinical outcome includes a reduction in tumor mass, a reduction in the rate of tumor growth, a reduction in metastasis, a reduction in the severity of the symptoms associated with the cancer and/or an increase in the longevity and/or quality of life of the subject compared with the absence of the treatment. In the case of inflammatory disorders and autoimmune diseases, a beneficial clinical outcome includes reduction in the extent or severity of the symptoms associated with the disease or disorder and/or an increase in the longevity and/or quality of life of the subject compared with the absence of the treatment. The precise amount of compound administered to a subject will depend on the type and severity of the disease or condition and on the characteristics of the subject, such as general health, age, sex, body weight and tolerance to drugs. It will also depend on the degree, severity and type of cancer, inflammatory disorder or autoimmune disease. The skilled artisan will be able to determine appropriate dosages depending on these and other factors. Effective amounts of the disclosed compounds typically range between about 1 mg/mm² per day and about 10 grams/mm² per day, and preferably between 10 mg/mm² per day and about 5 grams/mm².

The disclosed compounds may be administered by any suitable route, including, for example, orally in capsules, suspensions or tablets or by parenteral administration. Parenteral administration can include, for example, systemic administration, such as by intramuscular, intravenous, subcutaneous, or intraperitoneal injection. The compounds can also be administered orally (e.g., dietary inclusion), topically, by inhalation (e.g., intrabronchial, intranasal, oral inhalation or intranasal drops), or rectally, depending on the type of disease, disorder or condition to be treated. Oral and parenteral administration are preferred modes of administration.

The disclosed compounds can be administered to the subject in conjunction with an acceptable pharmaceutical carrier, adjuvant, diluent, excipient, solvent or other additives as part of a pharmaceutical composition. For convenience, the term “carrier” will encompass all such carriers, adjuvants, diluents, excipients, solvents or other inactive additives. Formulation of the compound to be administered will vary according to the route of administration selected (e.g., solution, emulsion, capsule) and the disease, disorder or condition targeted. Suitable pharmaceutical carriers may contain inert ingredients which do not substantially interact with the compound. Standard pharmaceutical formulation techniques can be employed, such as those described in Remington's Pharmaceutical Sciences, Mack Publishing Company, Easton, Pa. Suitable pharmaceutical carriers for parenteral administration include, for example, sterile water, physiological saline, bacteriostatic saline (saline containing about 0.9% mg/ml benzyl alcohol), phosphate-buffered saline, Hank's solution, Ringer's-lactate and the like. Methods for encapsulating compositions (such as in a coating of hard gelatin or cyclodextrasn) are known in the art (Baker, et al., “Controlled Release of Biological Active Agents”, John Wiley and Sons, 1986).

A “subject” is a mammal, preferably a human, but can also be an animal in need of veterinary treatment, e.g., companion animals (e.g., dogs, cats, and the like), farm animals (e.g., cows, sheep, pigs, horses, and the like) and laboratory animals (e.g., rats, mice, guinea pigs, and the like).

As used herein, a composition that “substantially” comprises a compound means that the composition contains more than about 80% by weight, more preferably more than about 90% by weight, even more preferably more than about 95% by weight, and most preferably more than about 97% by weight of the compound.

As used herein, a reaction that is “substantially complete” means that the reaction contains more than about 80% by weight of the desired product, more preferably more than about 90% by weight of the desired product, even more preferably more than about 95% by weight of the desired product, and most preferably more than about 97% by weight of the desired product.

As used herein, a racemic mixture means about 50% of one enantiomer and about 50% of is corresponding enantiomer relative to all chiral centers in the molecule. The invention encompasses all enantiomerically-pure, enantiomerically-enriched, diastereomerically pure, diastereomerically enriched, and racemic mixtures of the compounds of Formula (I).

Enantiomeric and diastereomeric mixtures can be resolved into their component enantiomers or stereoisomers by well known methods, such as chiral-phase gas chromatography, chiral-phase high performance liquid chromatography, crystallizing the compound as a chiral salt complex, or crystallizing the compound in a chiral solvent. Enantiomers and diastereomers can also be obtained from diastereomerically- or enantiomerically-pure intermediates, reagents, and catalysts by well known asymmetric synthetic methods.

Where a particular substituent occurs multiple times in a given structure (e.g., R₆ in Ring D), the identity of the substitutent is independent in each case and may be the same as or different from other occurrences of that substituent in the structure. Furthermore, individual substituents in the exemplary compounds shown below are preferred in combination with other substituents in the compounds of this invention, even if such exemplified substituents are not expressly noted as being preferred or not expressly shown in combination with other substituents.

The compounds of the invention are defined herein by their chemical structures and/or chemical names. Where a compound is referred to by both a chemical structure and a chemical name, and the chemical structure and chemical name conflict, the chemical structure is determinative of the compound's identity.

When administered to a patient, e.g., to a non-human animal for veterinary use or for improvement of livestock, or to a human for clinical use, the compounds of the invention are administered in isolated form or as the isolated form in a pharmaceutical composition. As used herein, “isolated” means that the compounds of the invention are separated from other components of either (a) a natural source, such as a plant or cell, preferably bacterial culture, or (b) a synthetic organic chemical reaction mixture. Preferably, via conventional techniques, the compounds of the invention are purified. As used herein, “purified” means that when isolated, the isolate contains at least about 90%, preferably at least about 95% or more preferably, at least about 98%, of a compound of this invention by weight of the isolate.

As used herein, a composition that is “substantially free” of a compound means that the composition contains less than about 20% by weight, more preferably less than about 10% by weight, even more preferably less than about 5% by weight, and most preferably less than about 3% by weight of the compound.

Choices and combinations of substituents and variables envisioned by this invention are only those that result in the formation of stable compounds. The term “stable”, as used herein, refers to compounds which possess stability sufficient to allow manufacture and which maintains the integrity of the compound for a sufficient period of time to be useful for the purposes detailed herein (e.g., therapeutic or prophylactic administration to a subject). Typically, such compounds are stable at a temperature of 40° C. or less, in the absence of excessive moisture, for at least one week. Such choices and combinations will be apparent to those of ordinary skill in the art and may be determined without undue experimentation.

This invention features indolizine compounds of formula (I):

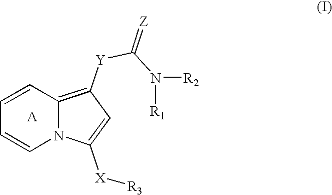

wherein Ring A is substituted or unsubstituted and is optionally fused to an aryl group;

Y is —C(R₄R₅)—, —N(R₄)—, —O—, —S—, —S(O)—, —S(O)₂—, —C(═O)—, —C(═S)—, —C(═O)—N(R₄)—, —C(═N—OR₁₂)—, —C(═N—R₁₂)—, or —N(R₄)—C(═O)—;

Z is ═O, ═S, ═N—OR₁₂ or ═NR₁₂;

R₁ and R₂ are independently —H, an unsubstituted aliphatic group, a substituted aliphatic group, an unsubstituted non-aromatic heterocylic group, a substituted non-aromatic heterocylic group, an unsubstituted aryl group or a substituted aryl group, provided that R₁ and R₂ are not both —H; or alternatively, NR₁R₂, taken together, is a substituted or unsubstituted non-aromatic nitrogen-containing heterocyclic group or a substituted or unsubstituted nitrogen-containing heteroaryl group R₃ is a substituted or unsubstituted aryl group or a substituted or unsubstituted aliphatic group;

X is a covalent bond, —C(R₄R₅)—, —N(R₄)—, —O—, —S—, —S(O)—, —S(O)₂—, —C(═O)—, —C(═O)—N(R₄)—, or —N(R₄)—C(═O)—;

each R₄ and R₅ is independently —H or a substituted or unsubstituted aliphatic group;

R₁₂ is —H or a substituted or unsubstituted alkyl group and pharmaceutically acceptable salts and prodrugs thereof.

One specific embodiment provides the compound of Formula (I) wherein Ring A is optionally substituted with halo, —C₁-C₄ alkyl, —C₁-C₄ alkoxy, —C₁-C₄ haloalkyl, C₁-C₄ haloalkoxy, —C₁-C₄ acyl, amido, substituted amido, —NO₂, —CN, —OH, —NH₂ and substituted amino; Y is —C(R₄R₅) or C═O; Z is ═O; R₁ is —H; R₂ is a substituted or unsubstituted alkyl group or a substituted or unsubstituted aryl group; R₃ is a substituted or unsubstituted aryl group; and X is —C(R₄R₅)—, —N(R₄)—, —C(O)— or —O—. In a more specific embodiment, Y is C═O; R₂ is an unsubstituted aryl group or an aryl group substituted with lower alkyl, amido, cyano, or halo; R₃ is a substituted or unsubstituted phenyl, a substituted or unsubstituted pyridyl or a substituted or unsubstituted thienyl; and X is —CH₂—, —CH(lower alkyl)-, —NH—, —N(lower alkyl)-, —C(O)— or —O—. For cancer therapy or prophylaxis, one embodiment includes compounds of Formula (I) wherein Y is not —C(═O).

In another specific embodiment, the compounds of formula (I) are those wherein Y is —C(R₄R₅) or C═O, Z is ═O; R₁ is —H; R₂ is a substituted or unsubstituted alkyl or aryl group; R₃ is a substituted or unsubstituted aryl group; X is —C(R₄R₅)—, —N(R₄)—, —C(O)— or —O— (preferably, X is —C(R₄R₅)—); and R₄, R₅, are as described above. More preferably, Z₁ is ═O; R₃ is a substituted or unsubstituted phenyl or pyridyl group; and R₄ and R₅ are both —H.

The indolizine compounds of this invention include the compounds of Formula (I) themselves, as well as their pharmaceutically acceptable salts and their prodrugs, if applicable. A salt, for example, can be formed between an anion and a positively charged substituent (e.g., amino) on an indolizine compound. Suitable anions include chloride, bromide, ioide, sulfate, nitrate, phosphate, citrate, methanesulfonate, trifluoroacetate, and acetate. Likewise, a salt can also be formed between a cation and a negatively charged substituent (e.g., carboxylate) on an indolizine compound of this invention. Suitable cations include sodium ion, potassium ion, magnesium ion, calcium ion, and an ammonium cation such as tetramethylamnonium ion. Examples of prodrugs include esters and other pharmaceutically acceptable derivatives, which, upon administration to a subject, are capable of providing active indolizine compounds.

In one embodiment, the compounds of Formula (I) are those of Formula (Ia):

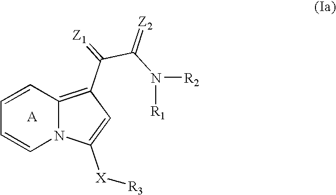

wherein Ring A is substituted or unsubstituted and is optionally fused to an aryl group;

Z₁ and Z₂ are independently ═O, ═S, ═N—OR₁₂ or ═NR₁₂;

R₁ and R₂ are independently —H, an unsubstituted aliphatic group, a substituted aliphatic group, an unsubstituted non-aromatic heterocylic group, a substituted non-aromatic heterocylic group, an unsubstituted aryl group or a substituted aryl group, provided that R₁ and R₂ are not both —H; or alternatively, NR₁R₂, taken together, is a substituted or unsubstituted non-aromatic nitrogen-containing heterocyclic group or a substituted or unsubstituted nitrogen-containing heteroaryl group R₃ is a substituted or unsubstituted aryl group or a substituted or unsubstituted aliphatic group;

X is a covalent bond, —C(R₄R₅)—, —N(R₄)—, —O—, —S—, —S(O)—, —S(O)₂—, —C(═O)—, —C(═O)—N(R₄)—, or —N(R₄)—C(═O)—;

each R₄ and R₅ are independently —H or a substituted or unsubstituted aliphatic group;

R₁₂ is —H or a substituted or unsubstituted alkyl group and pharmaceutically acceptable salts and prodrugs thereof.

One particular embodiment involves compounds of Formula (Ia) wherein Ring A is optionally substituted with halo, —C₁-C₄ alkyl, —C₁-C₄ alkoxy, —C₁-C₄ haloalkyl, C₁-C₄ haloalkoxy, —C₁-C₄ acyl, amido, substituted amido, —NO₂, —CN, —OH, —NH₂ and substituted amino (preferably, halo or lower alkyl), Z₁ and Z₂ are each ═O; R₁ is —H; R₂ is a substituted or unsubstituted aryl group or a substituted or unsubsituted non-aromatic heterocylic group; R₃ is a substituted or unsubstituted aryl group; and X is —C(R₄R₅)—, —N(R₄)— or —O—. In a more specific embodiment, R₂ is an unsubstituted aryl group or an aryl group substituted with lower alkyl, amido, cyano, or halo; R₃ is a substituted or unsubstituted phenyl, a substituted or unsubstituted pyridyl or a substituted or unsubstituted thienyl; and X is —CH₂—, —CH(lower alkyl)-, —NH—, —N(lower alkyl)- or —O—.

As noted above, values for R₁-R₃ include substituted and unsubstituted aryl groups. For these substituents (and particularly for R₂), aryl groups include those represented by Formulas (II)-(XV):

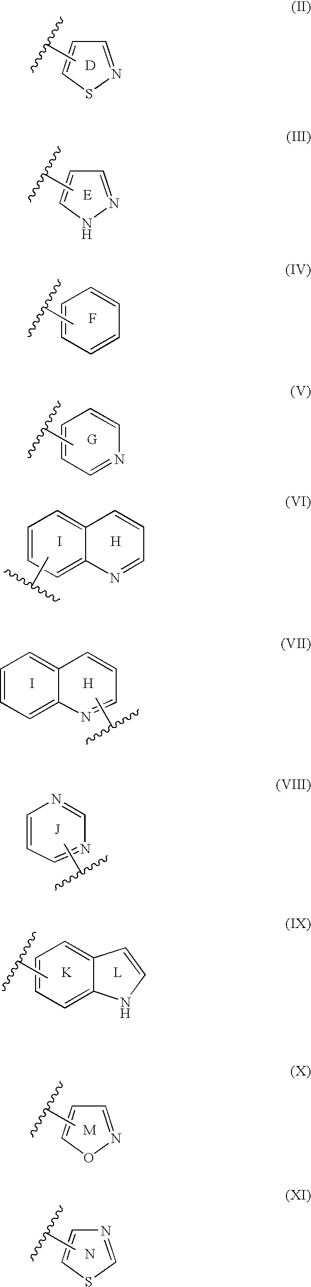

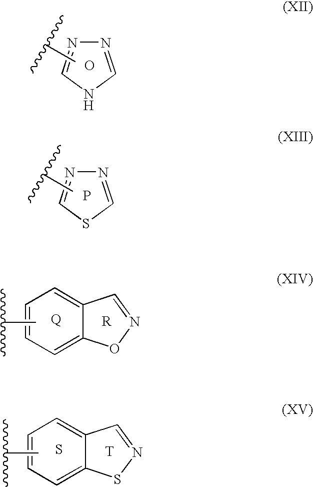

Rings D-T may be substituted or unsubstituted. Particular aryl groups for R₂ are represented by Formulas (XVI)-(XXI):

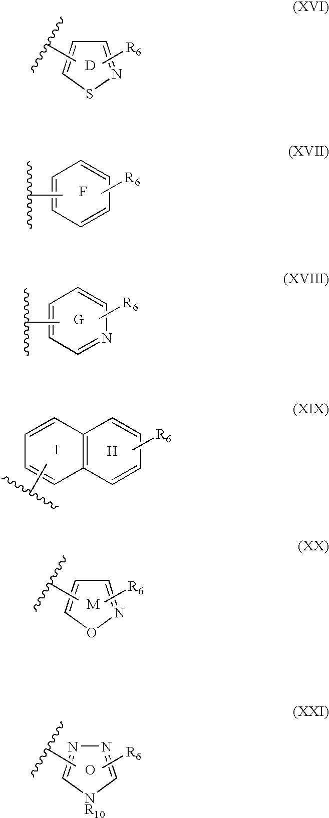

wherein R₆ occurs at each unfixed position in Rings D, F, G, I, H, M, and O, each R₆ is independently selected from the group consisting of H, C₁-C₄ alkyl, C₁-C₄ hydroxyalkyl, N-morpholino, pyrimidyl, C₁-C₄ alkyl substituted with pyrimidyl, —N(C1-C4 alkyl)₂, —C(O)NH₂, —C(O)NH(C₁-C₄ alkyl), C(O)N(C₁-C₄ alkyl)₂, —NHC(O)(C₁-C₄ alkyl), —NO₂, C₁-C₄ alkoxy, —C(O)O—CH₂CH₂—N(C₁-C₄ alkyl)₂,

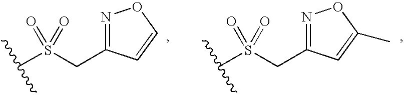

—NH—(phenyl), —NH₂, —CH₂NH—C(O)—O—(C₁-C₄ alkyl), —CH₂NH₂, —Cl, —F, —C(O)—O—(C₁-C₄ alkyl), —C(O)—N—(C₁-C₄ alkyl), C₃-C₇ cycloalkyl, phenyl, —C(O)—N-morpholino, —S—(C₁-C₄ alkyl), —CN, furyl, —S(O)₂—(C₁-C₄ alkyl), —S(O)₂—NH₂, —S(O)₂—NH(C₁-C₄ alkyl) or —S(O)₂—N(C₁-C₄ alkyl)₂ (alternatively, H, hydroxyl, cyano, nitro, halo, a substituted or unsubstituted alkyl group, a substituted or unsubstituted alkoxy group, or a substituted or unsubstituted aryl group), and R₁₀ is —H or a substutited or unsubstituted alkyl group.

Additional aryl groups for R₁, R₂, and R₃ (and in particular, R₂) are represented by Formulas (XXII)-(XXVII):

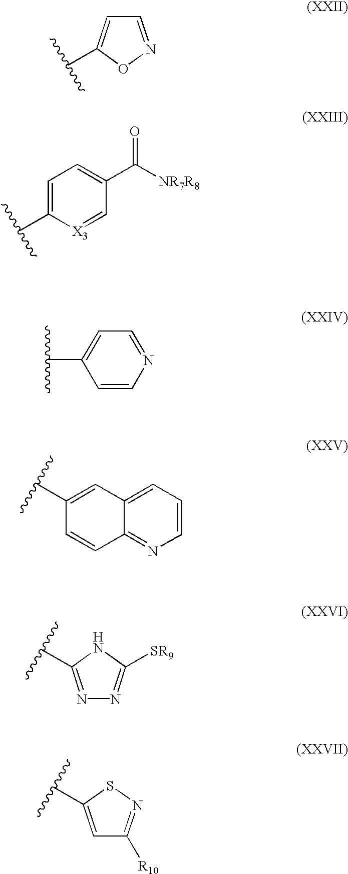

wherein X₃ is —CH— or —N—;

R₇ and R₈ are independently —H or an alkyl group or alternatively, —NR₇R₈, taken together, is a nitrogen-containing non-aromatic heterocyclic group;

R₉ is an alkyl group; and

R₁₀ is —H or a substituted or unsubstituted alkyl group.

In another embodiment, the compound of Formula (I) is a compound of Formula (Ib):

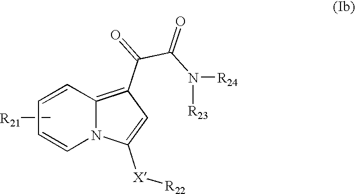

wherein R₂₁ occurs at each unfixed position of the ring system and each R₂₁ is independently H, lower alkyl, lower alkoxy, OH, F, Cl, Br, I, NO₂, or CN; R₂₂ is alkyl optionally substituted with lower alkoxy, OH, CN, F, Cl, Br, I, NO₂, NH₂, C(O)NH₂, CO₂H, or CO₂R′; or aryl optionally substituted with lower alkyl, lower alkoxy, OH, CN, F, Cl, Br, I, NO₂, NH₂, or C(O)NH₂, CO₂H, or CO₂R′; R₂₃ is H or lower alkyl; R₂₄ is N-oxy pyridyl or pyridyl optionally substituted with F, Cl, Br, or I; and X′ is C(R′R″), N(R′), O, S, S(O), S(O)₂, C(O), C(O)—N(R′), N(R′)—C(O), or deleted. Each of R′ and R″, independently, is H, or alkyl optionally substituted with lower alkoxy, OH, CN, F, Cl, Br, I, NO₂, NH₂, or C(O)NH₂.

A subset of the compounds of Formula (Ib) are those in which each R₂₁ is independently H, OH, F, or Cl; R₂₂ is phenyl optionally p-substituted with lower alkoxy, OH, CN, F, Cl, Br, I, NO₂, NH₂, C(O)NH₂, CO₂H, or CO₂R′; R₂₃ is H; R₂₄ is N-oxy p-pyridyl optionally substituted with F, Cl, Br, or I, or p-pyridyl also optionally substituted with one or more halogens; and X′ is CH₂. Another subset of the compounds covered by formulas (Ib) are those in which R₂₄ is o-pyridyl or m-pyridyl optionally substituted with F, Cl, Br, or I.

In another subset of compounds of Formula (I), the compounds of Formula (I) expressly exclude the compounds of Formula (Ia) as described above. Without wishing to be bound by theory, these compounds are TNFα and/or PDE4 inhibitors that can be used for preventing and treating inflammatory disorders, autoimmune diseases and other conditions involving PDE4 or elevated levels of cytokines, as well as use in the pharmaceutical compositions and methods described herein.

Shown below are exemplary compounds, compounds 1-64, of this invention:

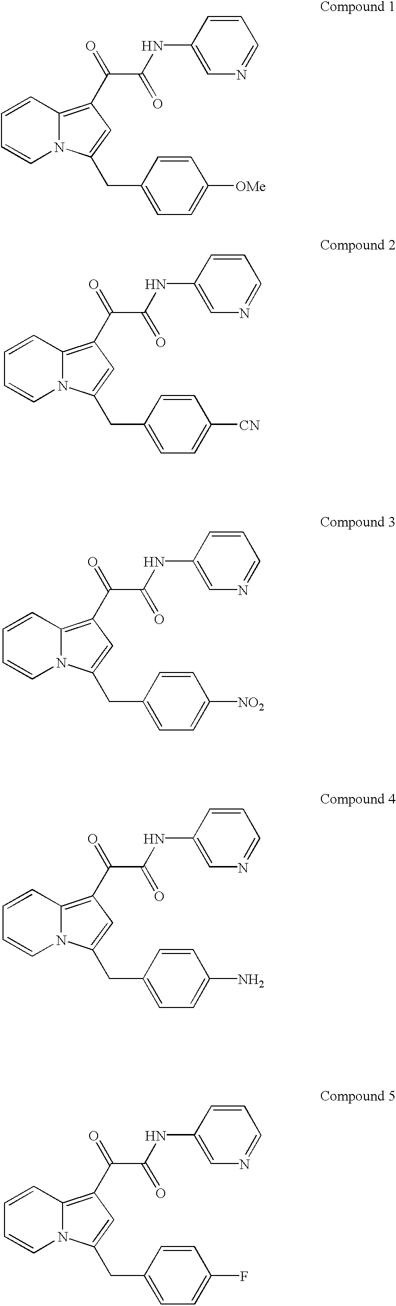

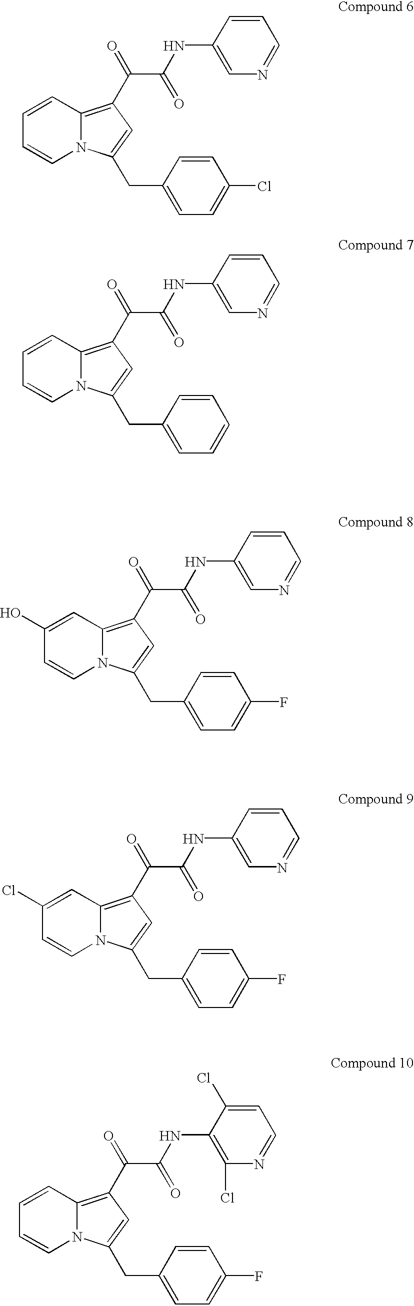

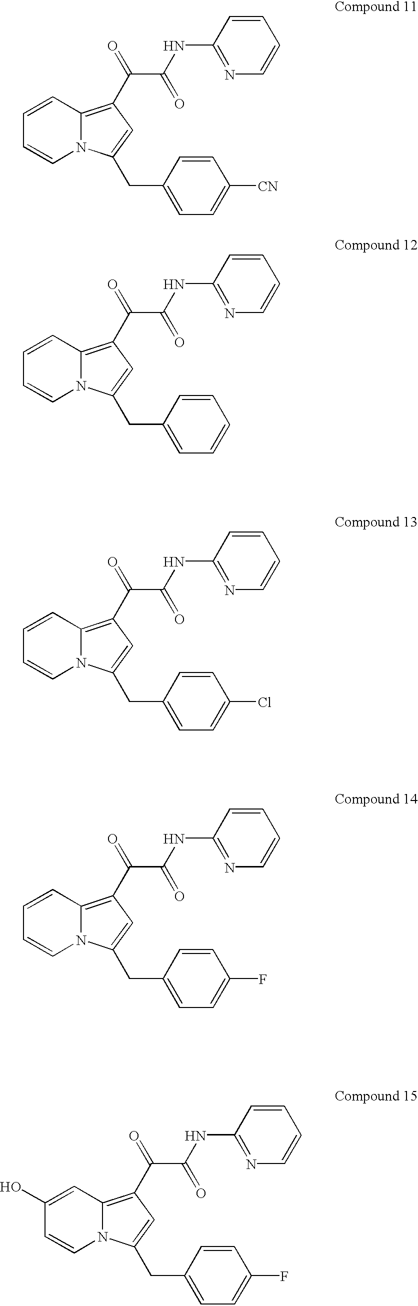

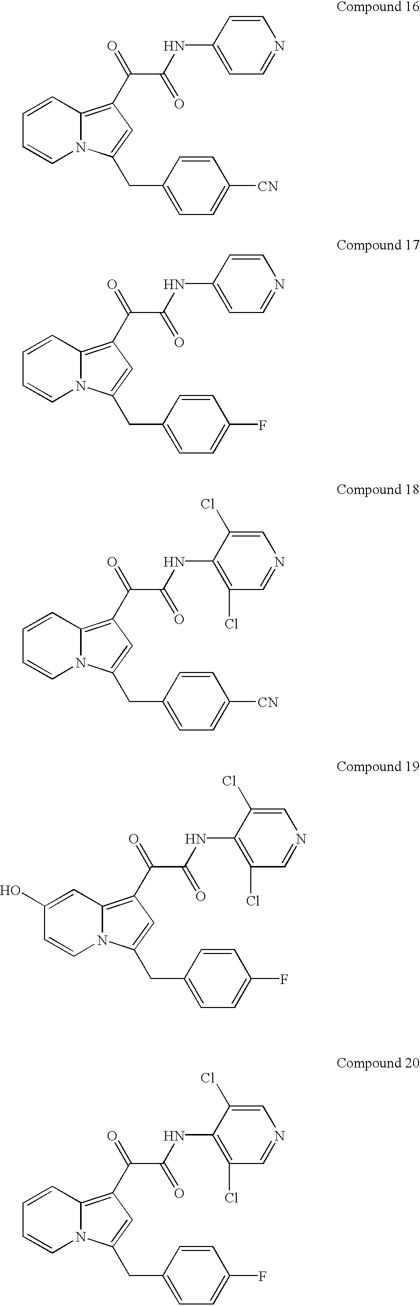

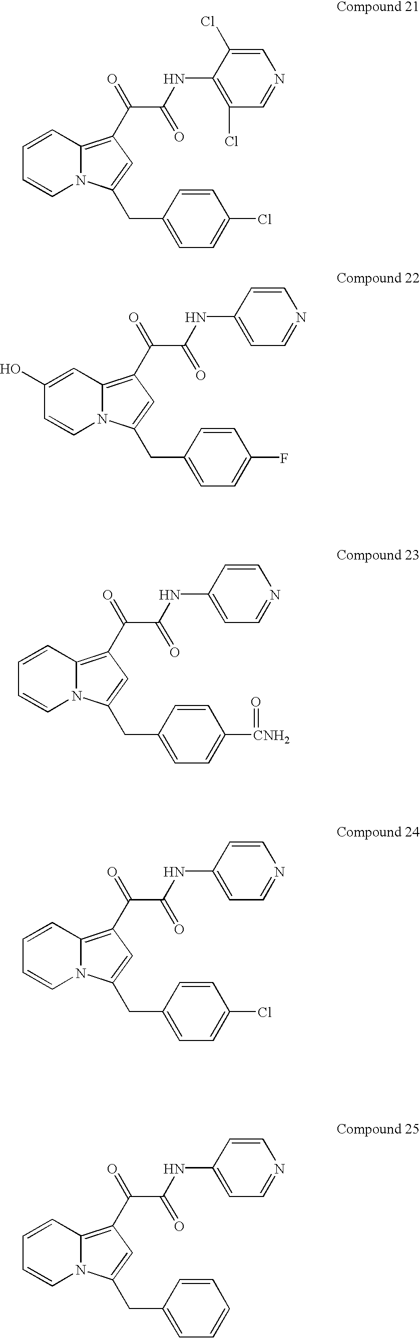

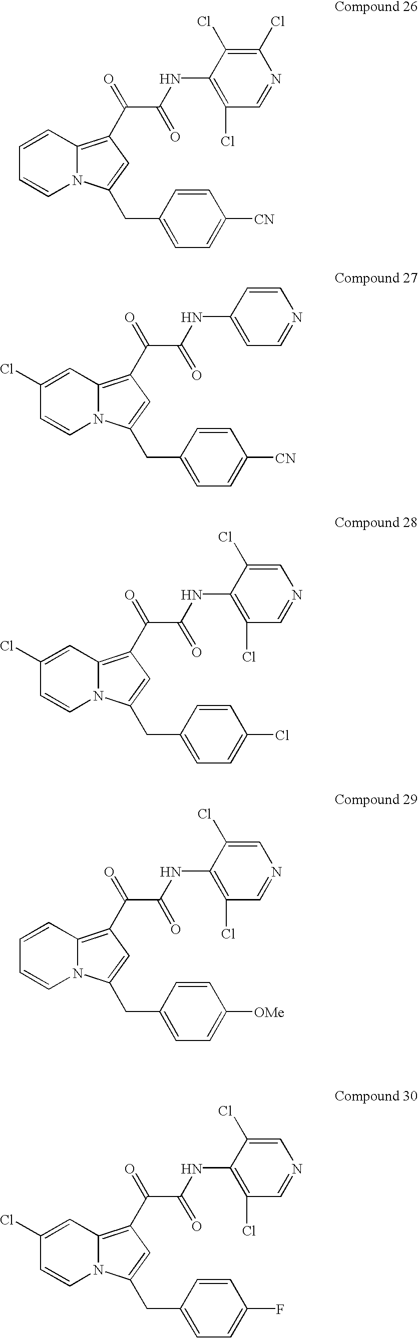

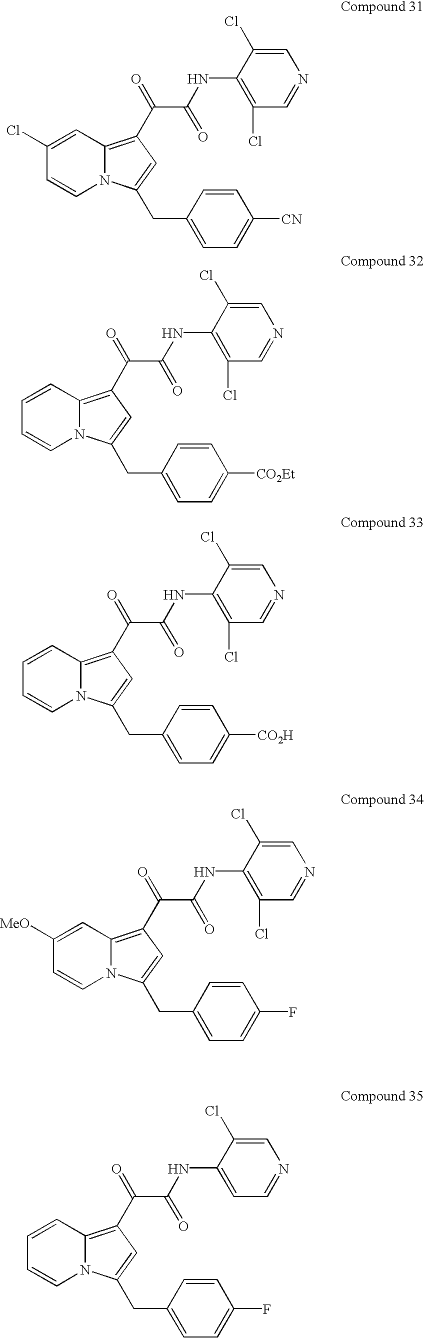

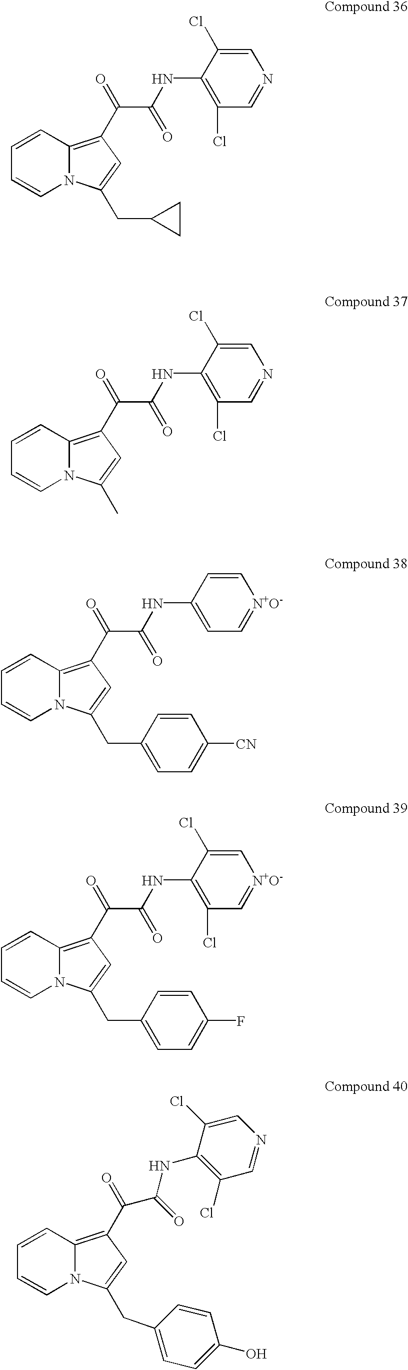

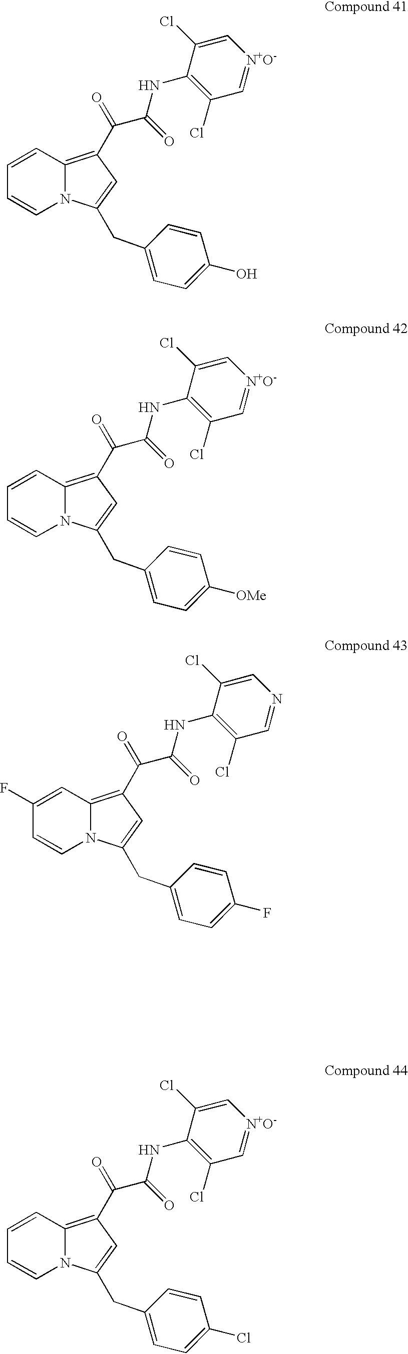

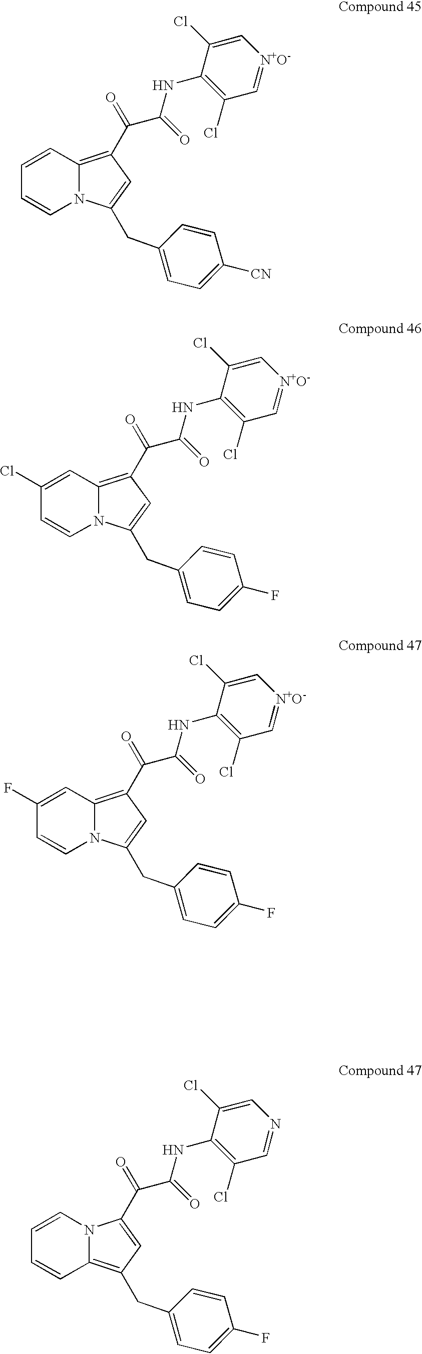

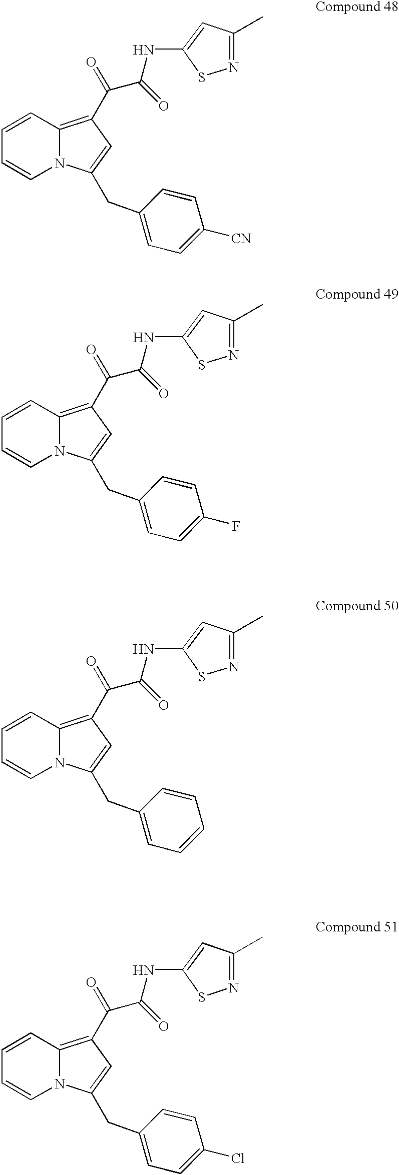

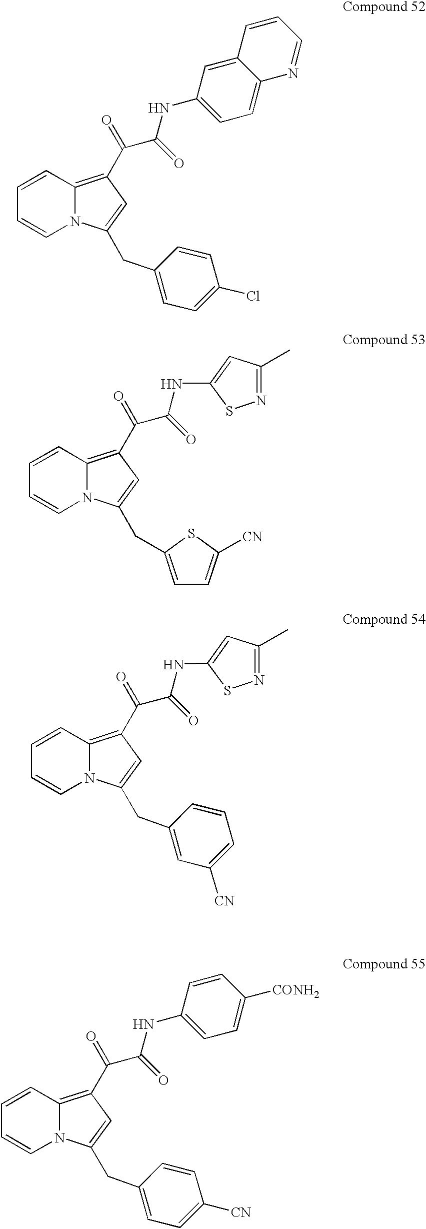

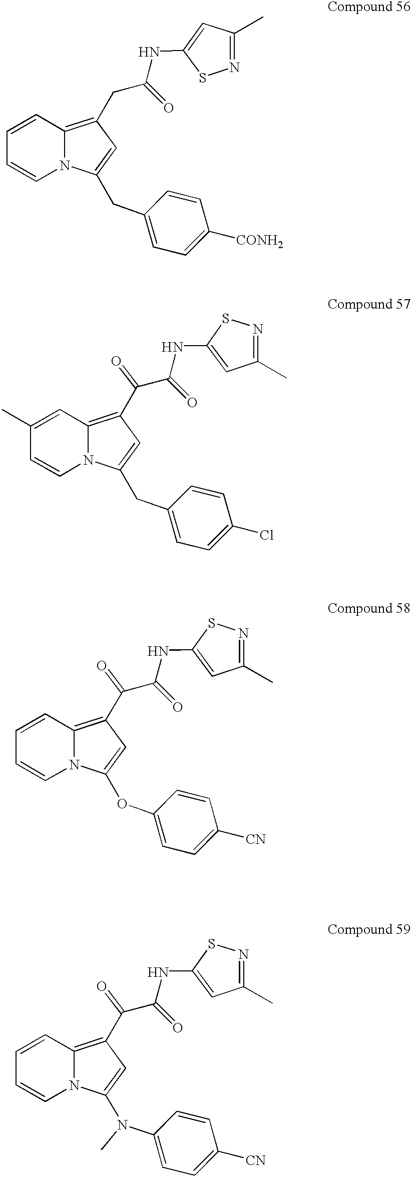

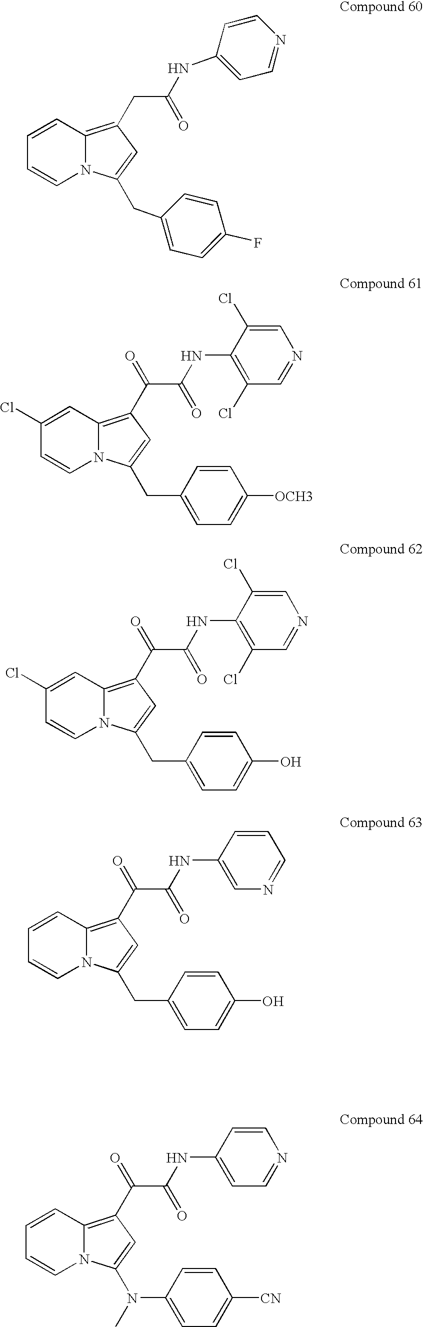

The scheme below depicts the syntheses of a large number of indolizine compounds of this invention, i.e., compounds 1-47 and 61-63. Details of preparation of compounds 1-47, 56, 58 and 60 are described in Examples 1-47, respectively. The synthesis of compounds 48-55, 57, 59, and 61-64 and related compounds is described in co-pending U.S. patent application Ser. No. 10/244,088 (the entire disclosure of which is hereby incorporated by reference). Based on these disclosures, other compounds of Formula (I) may be prepared by those of skill in the art without undue experimentation.

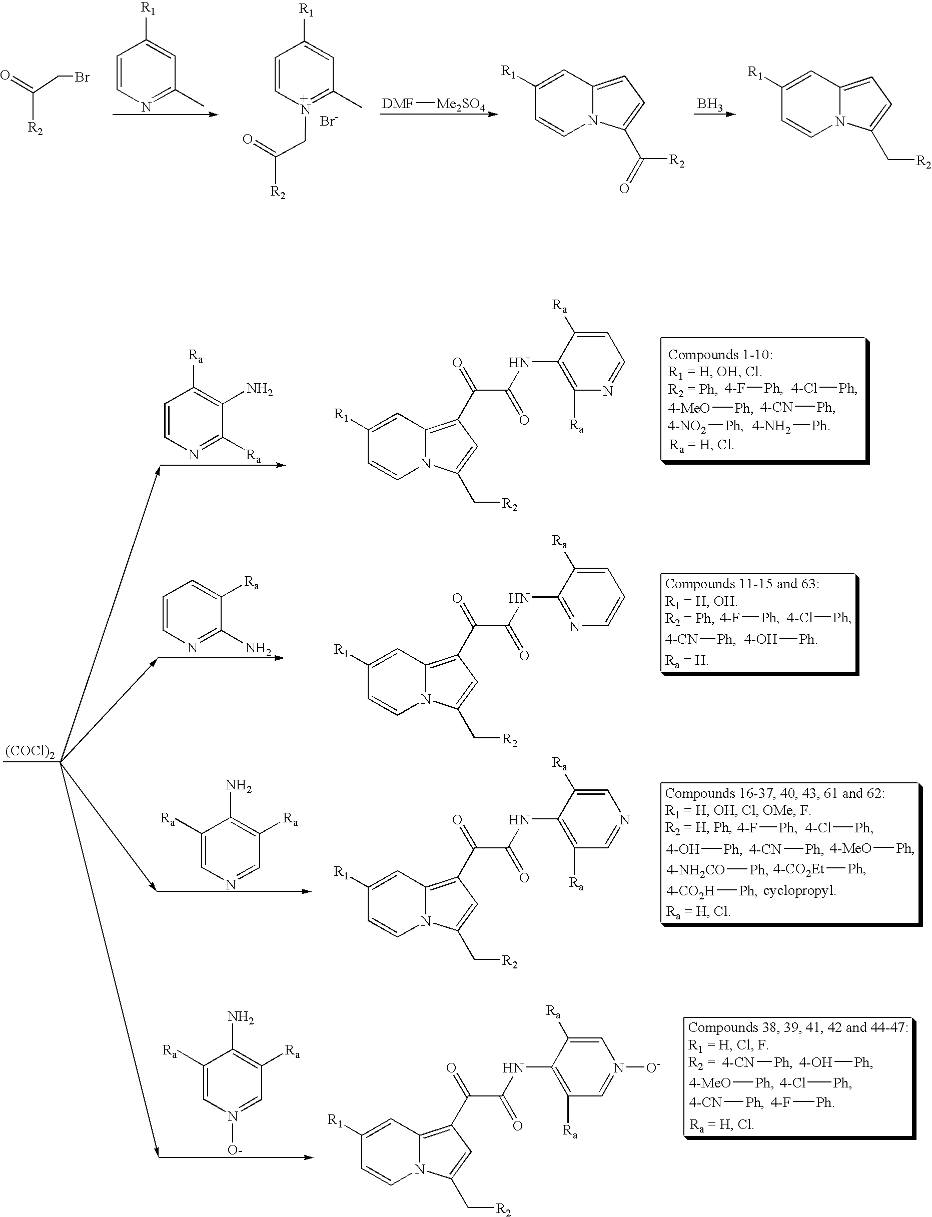

The indolizine compounds of this invention can be prepared by methods well known in the art, as well as by the synthetic routes disclosed herein. For example, one can react a 2-methylpyridine compound with a bromomethyl ketone compound to produce a pyridine salt. Treated with dimethyl sulfate, this pyridine salt forms an indolizine ring to give an indolizinyl ketone. This ketone can then be reduced to a 3-subsituted indolizine compound. A compound of this invention can be obtained by reacting the 3-substituted indolizine compound with 2-, 3-, or 4-aminopyridine or N-oxy 4-aminopyridine. Appropriate functional groups can be introduced into both the 2-methylpyridine compound and the aminopyridine compound. Any reactive groups on an indolizine intermediate can be protected prior to reacting the intermediate with an aminopyridine. For suitable protecting groups, see, e.g., Greene (1981) Protective Groups in Organic Synthesis, John Wiley & Sons, Inc., New York. An indolizine compound thus synthesized can be further purified by any conventional purification method, including without limitation, crystallization, flash column chromatography, solvating gas chromatography, or high performance liquid chromatography.

The indolizine compounds of the invention may contain a non-aromatic double bond and one or more asymmetric centers. Thus, they can occur as racemates and racemic mixtures, single enantiomers, individual diastereomers, diastereomeric mixtures, and cis- or trans-isomeric forms. All such isomeric forms are contemplated.

Also within the scope of this invention is a pharmaceutical composition contains an effective amount of at least one indolizine compound of the present invention and a pharmaceutical acceptable carrier. Further, this invention covers a method of administering an effective amount of one or more of the indolizine compounds described in the summary section above to an inflammatory disorder patient. “An effective amount” refers to the amount of an active indolizine compound that is required to confer a therapeutic effect on the treated subject. Effective doses will vary, as recognized by those skilled in the art, depending on the types of diseases treated, route of administration, excipient usage, and the possibility of co-usage with other therapeutic treatment.

To practice one method of the present invention, a composition having one or more indolizine compounds can be administered parenterally, orally, nasally, rectally, topically, or buccally. The term “parenteral” as used herein refers to subcutaneous, intracutaneous, intravenous, intrmuscular, intraarticular, intraarterial, intrasynovial, intrasternal, intrathecal, intralesional, or intracranial injection, as well as any suitable infusion technique.

A sterile injectable composition can be a solution or suspension in a non-toxic parenterally acceptable diluent or solvent, such as a solution in 1,3-butanediol. Among the acceptable vehicles and solvents that can be employed are mannitol, water, Ringer's solution, and isotonic sodium chloride solution. In addition, fixed oils are conventionally employed as a solvent or suspending medium (e.g., synthetic mono- or diglycerides). Fatty acid, such as oleic acid and its glyceride derivatives are useful in the preparation of injectables, as are natural pharmaceutically acceptable oils, such as olive oil or castor oil, especially in their polyoxyethylated versions. These oil solutions or suspensions can also contain a long chain alcohol diluent or dispersant, or carboxymethyl cellulose or similar dispersing agents. Other commonly used surfactants such as Tweens or Spans or other similar emulsifying agents or bioavailability enhancers which are commonly used in the manufacture of pharmaceutically acceptable solid, liquid, or other dosage forms can also be used for the purpose of formulation.

A composition for oral administration can be any orally acceptable dosage form including capsules, tablets, emulsions, and aqueous suspensions, dispersions, and solutions. In the case of tablets, commonly used carriers include lactose and corn starch. Lubricating agents, such as magnesium stearate, are also typically added. For oral administration in a capsule form, useful diluents include lactose and dried corn starch. When aqueous suspensions or emulsions are administered orally, the active ingredient can be suspended or dissolved in an oily phase combined with emulsifying or suspending agents. If desired, certain sweetening, flavoring, or coloring agents can be added. A nasal aerosol or inhalation composition can be prepared according to techniques well known in the art of pharmaceutical formulation. For example, such a composition can be prepared as a solution in saline, employing benzyl alcohol or other suitable preservatives, absorption promoters to enhance bioavailability, fluorocarbons, and/or other solubilizing or dispersing agents known in the art. A composition having one or more active indolizine compounds can also be administered in the form of suppositories for rectal administration.

The carrier in the pharmaceutical composition must be “acceptable” in the sense that it is compatible with the active ingredient of the composition (and preferably, capable of stabilizing the active ingredient) and not deleterious to the subject to be treated. One or more solubilizing agents can be utilized as pharmaceutical excipients for delivery of an active indolizine compound. Examples of other carriers include colloidal silicon oxide, magnesium stearate, cellulose, sodium lauryl sulfate, and D&C Yellow #10.

The indolizine compounds of this invention can be preliminarily screened for their efficacy in treating cancer, autoimmune diseases, inflammatory disorders or other conditions involving PDE4 or elevated levels of cytokines by one or more of the following in vitro assays (See Examples 65 and 66 below) and in vivo assays (See Examples 67, 68, and 69 below). Other methods will also apparent to those of ordinary skill in the art.

This invention encompasses kits which, when used by the medical practitioner, can simplify the administration of appropriate amounts of active ingredients to a subject. A typical kit of the invention comprises a unit dosage form of a compound of Formula (I), or a pharmaceutically acceptable prodrug or salt thereof, and a device that can be used to administer the active ingredient. Examples of such devices include, but are not limited to, syringes, drip bags, patches, and inhalers.

Kits of the invention can further comprise pharmaceutically acceptable vehicles that can be used to administer one or more active ingredients. For example, if an active ingredient is provided in a solid form that must be reconstituted for parenteral administration, the kit can comprise a sealed container of a suitable vehicle in which the active ingredient can be dissolved to form a particulate-free sterile solution that is suitable for parenteral administration. Examples of pharmaceutically acceptable vehicles for such use include, but are not limited to: Water for Injection USP; aqueous vehicles such as, but not limited to, Sodium Chloride Injection, Ringer's Injection, Dextrose Injection, Dextrose and Sodium Chloride Injection, and Lactated Ringer's Injection; water-miscible vehicles such as, but not limited to, ethyl alcohol, polyethylene glycol, and polypropylene glycol; and non-aqueous vehicles such as, but not limited to, corn oil, cottonseed oil, peanut oil, sesame oil, ethyl oleate, isopropyl myristate, and benzyl benzoate.

The methods for treating or preventing diseases, disorders and conditions according to this invention can further comprise administering to the subject an effective amount of one or more additional therapeutic agents. Such therapeutic agents may include those conventionally used to prevent or treat an autoimmune disease, inflammatory disorder, or cancer of interest. For example, other therapeutic agents may include, without limitation, steroids, non-steroidal anti-inflammatory agents, antihistamines, analgesics, anti-cancer agents and suitable mixtures thereof. In such combination therapy treatment, both the compounds of this invention and the other drug agent(s) are administered to mammals (e.g., humans, male or female) by conventional methods. The agents may be administered in a single dosage form or in separate dosage forms. Effective amounts of the other therapeutic agents are well known to those skilled in the art. However, it is well within the skilled artisan's purview to determine the other therapeutic agent's optimal effective-amount range. In one embodiment of the invention where another therapeutic agent is administered to a subject, the effective amount of the compound of this invention is less than its effective amount when the other therapeutic agent is not administered. In another embodiment, the effective amount of the conventional agent is less than its effective amount when the compound of this invention is not administered. In this way, undesired side effects associated with high doses of either agent may be minimized. Other potential advantages (including without limitation improved dosing regimens and/or reduced drug cost) will be apparent to those of skill in the art.

In the case of autoimmune and inflammatory conditions, the other therapeutic agent can be a steroid or a non-steroidal anti-inflammatory agent. Useful non-steroidal anti-inflammatory agents, include, but are not limited to, aspirin, ibuprofen, diclofenac, naproxen, benoxaprofen, flurbiprofen, fenoprofen, flubufen, ketoprofen, indoprofen, piroprofen, carprofen, oxaprozin, pramoprofen, muroprofen, trioxaprofen, suprofen, aminoprofen, tiaprofenic acid, fluprofen, bucloxic acid, indomethacin, sulindac, tolmetin, zomepirac, tiopinac, zidometacin, acemetacin, fentiazac, clidanac, oxpinac, mefenamic acid, meclofenamic acid, flufenamic acid, niflumic acid, tolfenamic acid, di flurisal, flufenisal, piroxicam, sudoxicam, isoxicam; salicylic acid derivatives, including aspirin, sodium salicylate, choline magnesium trisalicylate, salsalate, diflunisal, salicylsalicylic acid, sulfasalazine, and olsalazin; para-aminophennol derivatives including acetaminophen and phenacetin; indole and indene acetic acids, including indomethacin, sulindac, and etodolac; heteroaryl acetic acids, including tolmetin, diclofenac, and ketorolac; anthranilic acids (fenamates), including mefenamic acid, and meclofenamic acid; enolic acids, including oxicams (piroxicam, tenoxicam), and pyrazolidinediones (phenylbutazone, oxyphenthartazone); and alkanones, including nabumetone and pharmaceutically acceptable salts thereof and mixtures thereof. For a more detailed description of the NSAIDs, see Paul A. Insel, Analgesic-Antipyretic and Antiinflammatory Agents and Drugs Employed in the Treatment of Gout, in Goodman & Gilman's The Pharmacological Basis of Therapeutics 617-57 (Perry B. Molinhoff and Raymond W. Ruddon eds., 9^{th} ed 1996) and Glen R. Hanson, Analgesic, Antipyretic and Anti-Inflammatory Drugs in Remington: The Science and Practice of Pharmacy Vol II 1196-1221 (A. R. Gennaro ed. 19th ed. 1995) which are hereby incorporated by reference in their entireties.

Of particular relevance to allergic disorders, the other therapeutic agent can be an anthihistamine. Useful antihistamines include, but are not limited to, loratadine, cetirizine, fexofenadine, desloratadine, diphenhydramine, chlorpheniramine, chlorcyclizine, pyrilamine, promethazine, terfenadine, doxepin, carbinoxamine, clemastine, tripelennamine, brompheniramine, hydroxyzine, cyclizine, meclizine, cyproheptadine, phenindamine, acrivastine, azelastine, levocabastine, and mixtures thereof. For a more detailed description of anthihistamines, see Goodman & Gilman's The Pharmacological Basis of Therapeutics (2001) 651-57, 10^{th} ed).

In the case of cancer, the other therapeutic agent may be selected from any conventional anti-cancer agent appropriate for a target cancer. Examples of such anti-cancer agents include, without limitation, acivicin; aclarubicin; acodazole hydrochloride; acronine; adozelesin; aldesleukin; altretamine; ambomycin; ametantrone acetate; aminoglutethimide; amsacrine; anastrozole; anthramycin; asparaginase; asperlin; azacitidine; azetepa; azotomycin; batimastat; benzodepa; bicalutamide; bisantrene hydrochloride; bisnafide dimesylate; bizelesin; bleomycin sulfate; brequinar sodium; bropirimine; busulfan; cactinomycin; calusterone; caracemide; carbetimer; carboplatin; carmustine; carubicin hydrochloride; carzelesin; cedefingol; chlorambucil; cirolemycin; cisplatin; cladribine; crisnatol mesylate; cyclophosphamide; cytarabine; dacarbazine; dactinomycin; daunorubicin hydrochloride; decitabine; dexormaplatin; dezaguanine; dezaguanine mesylate; diaziquone; docetaxel; doxorubicin; doxorubicin hydrochloride; droloxifene; droloxifene citrate; dromostanolone propionate; duazomycin; edatrexate; eflomithine hydrochloride; elsamitrucin; enloplatin; enpromate; epipropidine; epirubicin hydrochloride; erbulozole; esorubicin hydrochloride; estramustine; estramustine phosphate sodium; etanidazole; etoposide; etoposide phosphate; etoprine; fadrozole hydrochloride; fazarabine; fenretinide; floxuridine; fludarabine phosphate; fluorouracil; flurocitabine; fosquidone; fostriecin sodium; gemcitabine; gemcitabine hydrochloride; hydroxyurea; idarubicin hydrochloride; ifosfamide; ilmofosine; interleukin II (including recombinant interleukin II, or rIL2), interferon alfa-2a; interferon alfa-2b; interferon alfa-n1; interferon alfa-n3; interferon beta-I a; interferon gamma-I b; iproplatin; irinotecan hydrochloride; lanreotide acetate; letrozole; leuprolide acetate; liarozole hydrochloride; lometrexol sodium; lomustine; losoxantrone hydrochloride; masoprocol; maytansine; mechlorethamine hydrochloride; megestrol acetate; melengestrol acetate; melphalan; menogaril; mercaptopurine; methotrexate; methotrexate sodium; metoprine; meturedepa; mitindomide; mitocarcin; mitocromin; mitogillin; mitomalcin; mitomycin; mitosper; mitotane; mitoxantrone hydrochloride; mycophenolic acid; nocodazole; nogalamycin; ormaplatin; oxisuran; paclitaxel; pegaspargase; peliomycin; pentamustine; peplomycin sulfate; perfosfamide; pipobroman; piposulfan; piroxantrone hydrochloride; plicamycin; plomestane; porfimer sodium; porfiromycin; prednimustine; procarbazine hydrochloride; puromycin; puromycin hydrochloride; pyrazofurin; riboprine; rogletimide; safingol; safingol hydrochloride; semustine; simtrazene; sparfosate sodium; sparsomycin; spirogermanium hydrochloride; spiromustine; spiroplatin; streptonigrin; streptozocin; sulofenur; talisomycin; tecogalan sodium; tegafur; teloxantrone hydrochloride; temoporfin; teniposide; teroxirone; testolactone; thiamiprine; thioguanine; thiotepa; tiazofurin; tirapazamine; toremifene citrate; trestolone acetate; triciribine phosphate; trimetrexate; trimetrexate glucuronate; triptorelin; tubulozole hydrochloride; uracil mustard; uredepa; vapreotide; verteporfin; vinblastine sulfate; vincristine sulfate; vindesine; vindesine sulfate; vinepidine sulfate; vinglycinate sulfate; vinleurosine sulfate; vinorelbine tartrate; vinrosidine sulfate; vinzolidine sulfate; vorozole; zeniplatin; zinostatin; zorubicin hydrochloride. Other anti-cancer drugs that may be used in combination therapy with the compounds of this invention include, but are not limited to: 20-epi-1,25 dihydroxyvitamin D3; 5-ethynyluracil; abiraterone; aclarubicin; acylfulvene; adecypenol; adozelesin; aldesleukin; ALL-TK antagonists; altretamine; ambamustine; amidox; amifostine; aminolevulinic acid; amrubicin; amsacrine; anagrelide; anastrozole; andrographolide; angiogenesis inhibitors; antagonist D; antagonist G; antarelix; anti-dorsalizing morphogenetic protein-1; antiandrogen, prostatic carcinoma; antiestrogen; antineoplaston; antisense oligonucleotides; aphidicolin glycinate; apoptosis gene modulators; apoptosis regulators; apurinic acid; ara-CDP-DL-PTBA; arginine deaminase; asulacrine; atamestane; atrimustine; axinastatin 1; axinastatin 2; axinastatin 3; azasetron; azatoxin; azatyrosine; baccatin III derivatives; balanol; batimastat; BCR/ABL antagonists; benzochlorins; benzoylstaurosporine; beta lactam derivatives; beta-alethine; betaclamycin B; betulinic acid; bFGF inhibitor; bicalutamide; bisantrene; bisaziridinylspermine; bisnafide; bistratene A; bizelesin; breflate; bropirimine; budotitane; buthionine sulfoximine; calcipotriol; calphostin C; camptothecin derivatives; canarypox IL-2; capecitabine; carboxamide-amino-triazole; carboxyamidotriazole; CaRest M3; CARN 700; cartilage derived inhibitor; carzelesin; casein kinase inhibitors (ICOS); castanospermine; cecropin B; cetrorelix; chlorlns; chloroquinoxaline sulfonamide; cicaprost; cis-porphyrin; cladribine; clomifene analogues; clotrimazole; collismycin A; collismycin B; combretastatin A4; combretastatin analogue; conagenin; crambescidin 816; crisnatol; cryptophycin 8; cryptophycin A derivatives; curacin A; cyclopentanthraquinones; cycloplatam; cypemycin; cytarabine ocfosfate; cytolytic factor; cytostatin; dacliximab; decitabine; dehydrodidemnin B; deslorelin; dexamethasone; dexifosfamide; dexrazoxane; dexverapamil; diaziquone; didemnin B; didox; diethylnorspermine; dihydro-5-azacytidine; dihydrotaxol, 9-; dioxamycin; diphenyl spiromustine; docetaxel; docosanol; dolasetron; doxifluridine; droloxifene; dronabinol; duocarmycin SA; ebselen; ecomustine; edelfosine; edrecolomab; eflomithine; elemene; emitefur;epirubicin; epristeride; estramustine analogue; estrogen agonists; estrogen antagonists; etanidazole; etoposide phosphate; exemestane; fadrozole; fazarabine; fenretinide; filgrastim; finasteride; flavopiridol; flezelastine; fluasterone; fludarabine; fluorodaunorunicin hydrochloride; forfenimex; formestane; fostriecin; fotemustine; gadolinium texaphyrin; gallium nitrate; galocitabine; ganirelix; gelatinase inhibitors; gemcitabine; glutathione inhibitors; hepsulfam; heregulin; hexamethylene bisacetamide; hypericin; ibandronic acid; idarubicin; idoxifene; idramantone; ilmofosine; ilomastat; imidazoacridones; imiquimod; immunostimulant peptides; insulin-like growth factor-1 receptor inhibitor; interferon agonists; interferons; interleukins; iobenguane; iododoxorubicin; ipomeanol, 4-; iroplact; irsogladine; isobengazole; isohomohalicondrin B; itasetron; jasplakinolide; kahalalide F; lamellarin-N triacetate; lanreotide; leinamycin; lenograstim; lentinan sulfate; leptoistatin; letrozole; leukemia inhibiting factor; leukocyte alpha interferon; leuprolide+estrogen+progesterone; leuprorelin; levamisole; liarozole; linear polyamine analogue; lipophilic disaccharide peptide; lipophilic platinum compounds; lissoclinamide 7; lobaplatin; lombricine; lometrexol; lonidamine; losoxantrone; lovastatin; loxoribine; lurtotecan; lutetium texaphyrin; lysofylline; lytic peptides; maitansine; mannostatin A; marimastat; masoprocol; maspin; matrilysin inhibitors; matrix metalloproteinase inhibitors; menogaril; merbarone; meterelin; methioninase; metoclopramide; MIF inhibitor; mifepristone; miltefosine; mirimostim; mismatched double stranded RNA; mitoguazone; mitolactol; mitomycin analogues; mitonafide; mitotoxin fibroblast growth factor-saporin; mitoxantrone; mofarotene; molgramostim; monoclonal antibody, human chorionic gonadotrophin; monophosphoryl lipid A+myobacterium cell wall sk; mopidamol; multiple drug resistance gene inhibitor; multiple tumor suppressor 1-based therapy; mustard anticancer agent; mycaperoxide B; mycobacterial cell wall extract; myriaporone; N-acetyldinaline; N-substituted benzamides; nafarelin; nagrestip; naloxone+pentazocine; napavin; naphterpin; nartograstim; nedaplatin; nemorubicin; neridronic acid; neutral endopeptidase; nilutamide; nisamycin; nitric oxide modulators; nitroxide antioxidant; nitrullyn; O6-benzylguanine; octreotide; okicenone; oligonucleotides; onapristone; ondansetron; ondansetron; oracin; oral cytokine inducer; ormaplatin; osaterone; oxaliplatin; oxaunomycin; paclitaxel; paclitaxel analogues; paclitaxel derivatives; palauamine; palmitoylrhizoxin; pamidronic acid; panaxytriol; panomifene; parabactin; pazelliptine; pegaspargase; peldesine; pentosan polysulfate sodium; pentostatin; pentrozole; perflubron; perfosfamide; perillyl alcohol; phenazinomycin; phenylacetate; phosphatase inhibitors; picibanil; pilocarpine hydrochloride; pirarubicin; piritrexim; placetin A; placetin B; plasminogen activator inhibitor; platinum complex; platinum compounds; platinum-triamine complex; porfimer sodium; porfiromycin; prednisone; propyl bis-acridone; prostaglandin J2; proteasome inhibitors; protein A-based immune modulator; protein kinase C inhibitor; protein kinase C inhibitors, microalgal; protein tyrosine phosphatase inhibitors; purine nucleoside phosphorylase inhibitors; purpurins; pyrazoloacridine; pyridoxylated hemoglobin polyoxyethylene conjugate; raf antagonists; raltitrexed; ramosetron; ras farnesyl protein transferase inhibitors; ras inhibitors; ras-GAP inhibitor; retelliptine demethylated; rhenium Re 186 etidronate; rhizoxin; ribozymes; RII retinamide; rogletimide; rohitukine; romurtide; roquinimex; rubiginone B1; ruboxyl; safingol; saintopin; SarCNU; sarcophytol A; sargramostim; Sdi 1 mimetics; semustine; senescence derived inhibitor 1; sense oligonucleotides; signal transduction inhibitors; signal transduction modulators; single chain antigen-binding protein; sizofiran; sobuzoxane; sodium borocaptate; sodium phenylacetate; solverol; somatomedin binding protein; sonermin; sparfosic acid; spicamycin D; spiromustine; splenopentin; spongistatin 1; squalamine; stem cell inhibitor; stem-cell division inhibitors; stipiamide; stromelysin inhibitors; sulfinosine; superactive vasoactive intestinal peptide antagonist; suradista; suramin; swainsonine; synthetic glycosaminoglycans; tallimustine; tamoxifen methiodide; tauromustine; tazarotene; tecogalan sodium; tegafur; tellurapyrylium; telomerase inhibitors; temoporfin; temozolomide; teniposide; tetrachlorodecaoxide; tetrazomine; thaliblastine; thiocoraline; thrombopoietin; thrombopoietin mimetic; thymalfasin; thymopoietin receptor agonist; thymotrinan; thyroid stimulating hormone; tin ethyl etiopurpurin; tirapazamine; titanocene bichloride; topsentin; toremifene; totipotent stem cell factor; translation inhibitors; tretinoin; triacetyluridine; triciribine; trimetrexate; triptorelin; tropisetron; turosteride; tyrosine kinase inhibitors; tyrphostins; UBC inhibitors; ubenimex; urogenital sinus-derived growth inhibitory factor; urokinase receptor antagonists; vapreotide; variolin B; vector system, erythrocyte gene therapy; velaresol; veramine; verdins; verteporfin; vinorelbine; vinxaltine; vitaxin; vorozole; zanoterone; zeniplatin; zilascorb; and zinostatin stimalamer. Preferred additional anti-cancer drugs are 5-fluorouracil and leucovorin. Examples of anti-cancer therapeutic antibodies that can be used in combination with the compounds of this invention include but are not limited to HERCEPTIN® (Trastuzumab) (Genentech, Calif.) which is a humanized anti-HER2 monoclonal antibody for the treatment of patients with metastatic breast cancer; REOPRO® (abciximab) (Centocor) which is an anti-glycoprotein IIb/IIIa receptor on the platelets for the prevention of clot formation; ZENAPAX® (daclizumab) (Roche Pharmaceuticals, Switzerland) which is an immunosuppressive, humanized anti-CD25 monoclonal antibody for the prevention of acute renal allograft rejection; PANOREX™ which is a murine anti-17-IA cell surface antigen IgG2a antibody (Glaxo Wellcome/Centocor); BEC2 which is a murine anti-idiotype (GD3 epitope) IgG antibody (ImClone System); IMC-C225 which is a chimeric anti-EGFR IgG antibody (ImClone System); VITAXIN™ which is a humanized anti-V3 integrin antibody (Applied Molecular Evolution/MedImmune); Campath 1H/LDP-03 which is a humanized anti CD52 IgG1 antibody (Leukosite); Smart M195 which is a humanized anti-CD33 IgG antibody (Protein Design Lab/Kanebo); RITUXAN™ which is a chimeric anti-CD20 IgG1 antibody (IDEC Pharm/Genentech, Roche/Zettyaku); LYMPHOCIDE™ which is a humanized anti-CD22 IgG antibody (Immunomedics); LYMPHOCIDE™ Y-90 (Immunomedics); Lymphoscan (Tc-99m-labeled; radioimaging; Immunomedics); Nuvion (against CD3; Protein Design Labs); CM3 is a humanized anti-ICAM3 antibody (ICOS Pharm); IDEC-114 is a primatied anti-CD80 antibody (IDEC Pharm/Mitsubishi); ZEVALIN™ is a radiolabelled murine anti-CD20 antibody (IDEC/Schering AG); IDEC-131 is a humanized anti-CD40L antibody (IDEC/Eisai); IDEC-151 is a primatized anti-CD4 antibody (IDEC); IDEC-152 is a primatized anti-CD23 antibody (IDEC/Seikagaku); SMART anti-CD3 is a humanized anti-CD3 IgG (Protein Design Lab); 5G1.1 is a humanized anti-complement factor 5 (C5) antibody (Alexion Pharm); D2E7 is a humanized anti-TNF-antibody (CAT/BASF); CDP870 is a humanized anti-TNF-Fab fragment (Celltech); IDEC-151 is a primatized anti-CD4 IgG1 antibody (IDEC Pharm/SmithKline Beecham); MDX-CD4 is a human anti-CD4 IgG antibody (Medarex/Eisai/Genmab); CD20-sreptdavidin (+biotin-yttrium 90; NeoRx); CDP571 is a humanized anti-TNF-IgG4 antibody (Celltech); LDP-02 is a humanized anti-47 antibody (LeukoSite/Genentech); OrthoClone OKT4A is a humanized anti-CD4 IgG antibody (Ortho Biotech); ANTOVA™ is a humanized anti-CD40L IgG antibody (Biogen); ANTEGREN™ is a humanized anti-VLA-4 IgG antibody (Elan); and CAT-152 is a human anti-TGF-2 antibody (Cambridge Ab Tech). Chemotherapeutic agents that can be used in the combination therapy methods and compositions of the invention include but are not limited to alkylating agents, antimetabolites, natural products, or hormones. Examples of alkylating agents useful for the treatment or prevention of particular cancers (especially those involving T-cell malignancies) include but are not limited to, nitrogen mustards (e.g., mechloroethamine, cyclophosphamide, chlorambucil, etc.), alkyl sulfonates (e.g., busulfan), nitrosoureas (e.g., carmustine, lomusitne, etc.), or triazenes (decarbazine, etc.). Examples of antimetabolites useful for the treatment or prevention of treatment or prevention of particular cancers (especially those involving T-cell malignancies) include but are not limited to folic acid analog (e.g., methotrexate), or pyrimidine analogs (e.g., Cytarabine), purine analogs (e.g., mercaptopurine, thioguanine, pentostatin). Examples of natural products useful for the treatment or prevention of treatment or prevention of particular cancers (especially those involving T-cell malignancies) include but are not limited to vinca alkaloids (e.g., vinblastin, vincristine), epipodophyllotoxins (e.g., etoposide), antibiotics (e.g., daunorubicin, doxorubicin, bleomycin), enzymes (e.g., L-asparaginase), or biological response modifiers (e.g., interferon alpha).

Examples of alkylating agents useful for the treatment or prevention of other cancers in the combination methods and compositions of the invention include but are not limited to, nitrogen mustards (e.g., mechloroethamine, cyclophosphamide, chlorambucil, melphalan, etc.), ethylenimine and methylmelamines (e.g., hexamethlymelamine, thiotepa), alkyl sulfonates (e.g., busulfan), nitrosoureas (e.g., carmustine, lomusitne, semustine, streptozocin, etc.), or triazenes (decarbazine, etc.). Examples of antimetabolites useful for the treatment or prevention of other cancers in the combination methods and compositions of the invention include but are not limited to folic acid analog (e.g., methotrexate), or pyrimidine analogs (e.g., fluorouracil, floxouridine, Cytarabine), purine analogs (e.g., mercaptopurine, thioguanine, pentostatin). Examples of natural products useful for the treatment or prevention of other cancers in the combination methods and compositions of the invention include but are not limited to vinca alkaloids (e.g., vinblastin, vincristine), epipodophyllotoxins (e.g., etoposide, teniposide), antibiotics (e.g., actinomycin D, daunorubicin, doxorubicin, bleomycin, plicamycin, mitomycin), enzymes (e.g., L-asparaginase), or biological response modifiers (e.g., interferon alpha). Examples of hormones and antagonists useful for the treatment or prevention of other cancers in the combination methods and compositions of the invention include but are not limited to adrenocorticosteroids (e.g., prednisone), progestins (e.g., hydroxyprogesterone caproate, megestrol acetate, medroxyprogesterone acetate), estrogens (e.g., diethlystilbestrol, ethinyl estradiol), antiestrogen (e.g., tamoxifen), androgens (e.g., testosterone propionate, fluoxymesterone), antiandrogen (e.g., flutamide), gonadotropin releasing hormone analog (e.g., leuprolide). Other anti-cancer agents that can be used in the combination methods and compositions of the invention for the treatment or prevention of cancer include platinum coordination complexes (e.g., cisplatin, carboblatin), anthracenedione (e.g., mitoxantrone), substituted urea (e.g., hydroxyurea), methyl hydrazine derivative (e.g., procarbazine), adrenocortical suppressant (e.g., mitotane, aminoglutethimide).

The compounds of this invention may also be administered in combination with anti-cancer agents which act by arresting cells in the G2-M phases due to stabilized microtubules. In addition to Taxol (paclitaxel), and analogs and derivatives thereof, other examples of anti-cancer agents which act by this mechanism include without limitation the following marketed drugs and drugs in development: Erbulozole (also known as R-55104), Dolastatin 10 (also known as DLS-10 and NSC-376128), Mivobulin isethionate (also known as CI-980), Vincristine, NSC-639829, Discodermolide (also known as NVP-XX-A-296), ABT-751 (Abbott, also known as E-7010), Altorhyrtins (such as Altorhyrtin A and Altorhyrtin C), Spongistatins (such as Spongistatin 1, Spongistatin 2, Spongistatin 3, Spongistatin 4, Spongistatin 5, Spongistatin 6, Spongistatin 7, Spongistatin 8, and Spongistatin 9), Cemadotin hydrochloride (also known as LU-103793 and NSC-D-669356), Epothilones (such as Epothilone A, Epothilone B, Epothilone C (also known as desoxyepothilone A or dEpoA), Epothilone D (also referred to as KOS-862, dEpoB, and desoxyepothilone B), Epothilone E, Epothilone F, Epothilone B N-oxide, Epothilone A N-oxide, 16-aza-epothilone B, 21-aminoepothilone B (also known as BMS-310705), 21-hydroxyepothilone D (also known as Desoxyepothilone F and dEpoF), 26-fluoroepothilone), Auristatin PE (also known as NSC-654663), Soblidotin (also known as TZT-1027), LS-4559-P (Pharmacia, also known as LS-4577), LS-4578 (Pharmacia, also known as LS-477-P), LS-4477 (Pharmacia), LS-4559 (Pharmacia), RPR-112378 (Aventis), Vincristine sulfate, DZ-3358 (Daiichi), FR-182877 (Fujisawa, also known as WS-9885B), GS-164 (Takeda), GS-198 (Takeda), KAR-2 (Hungarian Academy of Sciences), BSF-223651 (BASF, also known as ILX-651 and LU-223651), SAH-49960 (Lilly/Novartis), SDZ-268970 (Lilly/Novartis), AM-97 (Armad/Kyowa Hakko), AM-132 (Armad), AM-138 (Ar mnad/Kyowa Hakko), IDN-5005 (Indena), Cryptophycin 52 (also known as LY-355703), AC-7739 (Ajinomoto, also known as AVE-8063A and CS-39.HCl), AC-7700 (Ajinomoto, also known as AVE-8062, AVE-8062A, CS-39-L-Ser.HCl, and RPR-258062A), Vitilevuamide, Tubulysin A, Canadensol, Centaureidin (also known as NSC-106969), T-138067 (Tularik, also known as T-67, TL-138067 and TI-138067), COBRA-1 (Parker Hughes Institute, also known as DDE-261 and WHI-261), H10 (Kansas State University), H16 (Kansas State University), Oncocidin A1 (also known as BTO-956 and DIME), DDE-313 (Parker Hughes Institute), Fijianolide B, Laulimalide, SPA-2 (Parker Hughes Institute), SPA-1 (Parker Hughes Institute, also known as SPIKET-P), 3-IAABU (Cytoskeleton/Mt. Sinai School of Medicine, also known as MF-569), Narcosine (also known as NSC-5366), Nascapine, D-24851 (Asta Medica), A-105972 (Abbott), Hemiasterlin, 3-BAABU (Cytoskeleton/Mt. Sinai School of Medicine, also known as MF-191), TMPN (Arizona State University), Vanadocene acetylacetonate, T-138026 (Tularik), Monsatrol, Inanocine (also known as NSC-698666), 3-IAABE (Cytoskeleton/Mt. Sinai School of Medicine), A-204197 (Abbott), T-607 (Tularik, also known as T-900607), RPR-115781 (Aventis), Eleutherobins (such as Desmethyleleutherobin, Desaetyleleutherobin, Isoeleutherobin A, and Z-Eleutherobin), Caribaeoside, Caribaeolin, Halichondrin B, D-64131 (Asta Medica), D-68144 (Asta Medica), Diazonamide A, A-293620 (Abbott), NPI-2350 (Nereus), Taccalonolide A, TUB-245 (Aventis), A-259754 (Abbott), Diozostatin, (-)-Phenylahistin (also known as NSCL-96F037), D-68838 (Asta Medica), D-68836 (Asta Medica), Myoseverin B, D-43411 (Zentaris, also known as D-81862), A-289099 (Abbott), A-318315 (Abbott), HTI-286 (also known as SPA-110, trifluoroacetate salt) (Wyeth), D-82317 (Zentaris), D-82318 (Zentaris), SC-12983 (NCI), Resverastatin phosphate sodium, BPR-OY-007 (National Health Research Institutes), and SSR-250411 (Sanofi).

In any case where pain in a component of the target disorder, the other therapeutic agent can be an analgesic. Useful analgesics include, but are not limited to, phenacetin, butacetin, acetaminophen, nefopam, acetoamidoquinone, and mixtures thereof.

The foregoing and other useful combination therapies will be understood and appreciated by those of skill in the art. Potential advantages of such combination therapies include the ability to use less of each of the individual active ingredients to minimize toxic side effects, synergistic improvements in efficacy, improved ease of administration or use and/or reduced overall expense of compound preparation or formulation.

The compounds of this invention may be used as research tools (for example, as a positive control to evaluate the mechanism of new TNFα or PDE4 inhibitors by competitive binding assays or to isolate ligands of the compounds of this invention using affinity chromatography. These and other uses and embodiments of the compounds and compositions of this invention will be apparent to those of ordinary skill in the art.

The invention is further defined by reference to the following examples describing in detail the preparation of compounds of the invention. It will be apparent to those skilled in the art that many modifications, both to materials and methods, may be practiced without departing from the purpose and interest of this invention. The following examples are set forth to assist in understanding the invention and should not be construed as specifically limiting the invention described and claimed herein. Such variations of the invention, including the substitution of all equivalents now known or later developed, which would be within the purview of those skilled in the art, and changes in formulation or minor changes in experimental design, are to be considered to fall within the scope of the invention incorporated herein. The specific examples below are to be construed as merely illustrative, and not limitative of the remainder of the disclosure in any way whatsoever. Without further elaboration, it is believed that one skilled in the art can, based on the description herein, utilize the present invention to its fullest extent. All publications cited herein are hereby incorporated by reference in their entirety.

### EXAMPLE 1

### Preparation of Compound 1: 2-[3-(4-methoxy-benzyl)-indolizin-1-yl]-2-oxo-N-pyridin-3-yl-acetamide

Picoline (2.18 mL, 22 mmol) was added to a stirred solution of 2-bromo-1-(4-methoxy-phenyl)-ethanone (2.5 g, 10.9 mmol) in 11 mL of acetonitrile at room temperature. The solution was continually stirred at room temperature for 2 hours. 15 mL of ethyl acetate was then added to the above solution. The resultant precipitate was collected by filtration and washed with ethyl acetate to give intermediate 1 as a white solid (2.4 g, 68%).

To a stirred suspension of intermediate 1 (2.4 g, 7.45 mmol) in 22 mL of dimethylformamide (DMF) was added 18 mL of DMF-Me₂SO₄ (obtained by stirring a mixture of 1 eq. DMF and 1 eq. Me₂SO₄ at 60-80° C. for 3 hours, followed by cooling to room temperature). Stirring was continued at room temperature for another 15 minutes. 31 mL of triethylamine was added to the above suspension, followed by stirring at 40-50° C. (reaction temperature) for 2 hours. After cooled to room temperature, the mixture was poured into 100 mL of ice water and was stirred for several hours. The resultant precipitate was collected, washed with water, and dried to give of intermediate 2 as an orange solid (1.4 g, 75%).

BH₃-THF (1M, 26 mL) was added to a solution of intermediate 2 (1.4 g, 11.2 mmol) in 33 mL of acetonitrile containing 0.5 mL of methanol. The resulting solution was stirred at 50° C. for 1 hour. The reaction mixture was cooled to ˜10° C. and quenched with 4 mL of ice water. 20 mL of ethyl acetate was added to the mixture, followed by drying with anhydrous Na₂SO₄. The solution was then decanted and evaporated under reduced pressure. The crude product was purified by solvating gas chromatography (SGC) using a gradient elution (hexane to 8:1 hexane/dichloromethane to 1:1 hexane/dichloromethane) to give intermediate 3 as an off-white solid (0.6 g, 43%).

A 10 mL dry ether solution containing intermediate 3 (0.39 g, 1.65 mmol) was slowly added to a stirred solution of oxalyl chloride (0.17 mL, 1.98 mmol) in 5 mL of dry ether at 0° C. After stirred at the same temperature for 30 minutes, the solution was concentrated to yield a solid and the resultant solid was re-dissolved in 5 mL of dry THF. A solution of 3-aminopyridine (0.37 g, 3.95 mmol) in 910 mL of dry THF was then added slowly to the above solution at 0° C. Stirring was continued at 0° C. for 1 hour and at room temperature for 2.5 hours. The volatiles were then removed under reduced pressure and the residue was dissolved in 30 mL of ethyl acetate. The ethyl acetate solution was washed successively with H₂O, saturated NaHCO₃, and brine. After dried with Na₂SO₄, the solvent of the solution was removed. The crude product was purified by SGC using a gradient elution (hexane to 2:1 hexane/ethyl acetate to 1:1 hexane/ethyl acetate) to give compound 1 as a yellow solid (0.3 g, 47%).

¹H NMR (CDCl₃) δ(ppm): 3.65 (s, 3H); 4.10 (s, 2H); 6.60-6.82 (m, 3H); 7.15 (q, J=7 Hz, 2H); 7.19-7.24 (m, 2H); 7.78 (d, J=7 Hz, 1H); 7.97 (s, 1H); 8.18-8.24 (m, 1H); 8.32 (dd, J=1.5 Hz, 5 Hz, 1H); 8.52 (d, J=9 Hz, 1H); 8.74 (d, J=2.4 Hz, 1H,); 9.55 (s, 1H)

ESMS calculated for (C₂₃H₁₉N₃O₃): 385.1; found: 386.1 (M+H)⁺

### EXAMPLE 2

### Preparation of Compound 2: 2-[3-(4-cyano-benzyl)-indolizin-1-yl]-2-oxo-N-pyridin-3-yl-acetamide

Compound 2 was prepared in a manner similar to that described in Example 1.

¹H-NMR (CDCl₃) δ(ppm): 9.52 (s, 1H); 8.81 (d, J=4.8 Hz, 1H); 8.66 (d, J=12 Hz, 1H); 8.45-8.26 (m, 2H); 8.13 (s, 1H); 7.81 (d, J=12 Hz, 1H); 7.64-7.30 (m, 6H); 6.96 (m, 1H); 4.26 (s, 2H)

ESMS calculated for (C₂₃H₁₆N₄O₂): 380.13; Found: 381.2 (M+H)⁺

### EXAMPLE 3

### Preparation of Compound 3: 2-[3-(4-nitro-benzyl)-indolizin-1-yl]-2-oxo-N-pyridin-3-yl-acetamide

Compound 3 was prepared in a manner similar to that described in Example 1.

¹H NMR (CDCl₃) δ(ppm): 4.25 (s, 2H); 6.83 (t, J=7 Hz, 1H); 7.14-7.32 (m, 4H); 7.71 (d, J=7 Hz, 1H); 7.97 (s, 1H); 8.03 (d, J=8 Hz, 2H); 8.17 (dt, J=8 Hz, 1.5 Hz, 1H); 8.28 (d, J=4.8 Hz, 1H); 8.48 (dd, J=9 Hz, 1.2 Hz, 1H); 8.74 (s, 1H); 9.59 (s, 1H)

ESMS calculated for (C₂₂H₁₆N₄O₄): 400.1; found: 401.1 (M+H)⁺

### EXAMPLE 4

### Preparation of Compound 4: 2-[3-(4-amino-benzyl)-indolizin-1-yl]-2-oxo-N-pyridin-3-yl-acetamide

Compound 4 was prepared in a manner similar to that described in Example 1.

¹H NMR (CDCl₃) δ(ppm): 3.65 (bs, 2H); 4.18 (s, 2H); 6.63 (d, J=9 Hz, 1H); 6.9 (t, J=7 Hz, 1H); 6.98 (d, J=7 Hz, 2H); 7.28-7.39 (m, 2H); 7.90 (d, J=7 Hz, 1H); 8.06 (s, 1H); 8.28-8.34 (m, 1H); 8.41 (dd, J=5 Hz, 1.5 Hz, 1H); 8.63 (d, J=9 Hz, 1H); 8.83 (d, J=2.4 Hz, 1H); 9.62 (s, 1H)

ESMS calculated for (C₂₂H₁₈N₄O₂): 370.1; found: 371.1 (M+H)⁺

### EXAMPLE 5

### Preparation of Compound 5: 2-[3-(4-fluoro-benzyl)-indolizin-1-yl]-2-oxo-N-pyridin-3-yl-acetamide

Compound 5 was prepared in a manner similar to that described in Example 1.

¹H NMR (CDCl₃) δ(ppm): 9.53 (s, 1H); 8.81 (d, J=3.0 Hz, 1H); 8.66 (d, J=9.0 Hz, 1H); 8.40 (d, J=6.0 Hz, 2H); 8.30 (s, 1H); 7.85 (d, J=9.0 Hz, 1H); 7.35 (m, 3H); 7.96 (m, 3H); 4.24 (s, 2H)

ESMS calculated (C₂₂H₁₆FN₃O₂): 373.1; found: 374.1 (M+H)⁺

### EXAMPLE 6

### Preparation of Compound 6: 2-[3-(4-chloro-benzyl)-indolizin-1-yl]-2-oxo-N-pyridin-3-yl-acetamide

Compound 6 was prepared in a manner similar to that described in Example 1.

¹H NMR (CDCl₃) δ(ppm): 9.53 (s, 1H); 8.81 (d, J=3.0 Hz, 1H); 8.66 (d, J=9.0 Hz 1H); 8.42 (d, J=6.0 Hz, 1H); 8.30 (m, 1H); 8.11 (s, 1H); 7.83 (d, J=6.0 Hz, 1H); 7.36 (m, 3H); 7.15 (d, J=9.0 Hz, 2H); 6.93 (m, 1H); 4.24 (s, 2H)

ESMS calculated (C₂₂H₁₆CIN₃O₂): 389.1; found: 390.1 (M+H)⁺

### EXAMPLE 7

### Preparation of Compound 7: 2-(3-benzyl-indolizin-1-yl)-2-oxo-N-pyridin-3-yl-acetamide

Compound 7 was prepared in a manner similar to that described in Example 1.

¹H NMR (CDCl₃) δ(ppm): 4.20 (s, 2H); 6.82 (t, J=7 Hz, 1H); 7.11-7.32 (m, 7H); 7.80 (d, J=7 Hz, 1H); 8.04 (s, 1H); 8.22-8.27 (m, 1H); 8.34 (dd, J=1.4 Hz, 5 Hz, 1H); 8.56 (d, J=9 Hz, 1H); 8.74 (d, J=3 Hz, 1H); 9.49 (s, 1H)

ESMS calculated for (C₂₂H₁₇N₃O₂): 355.1; found: 356.1 (M+H)⁺

### EXAMPLE 8

### Preparation of Compound 8: 2-[3-(4-fluoro-benzyl)-7-hydroxy-indolizin-1-yl]-2-oxo-N-pyridin-3-yl-acetamide

3.2 g (60%, 80 mmol) of NaH was added to a 130 mL DMF solution containing 8.88 g (82 mmol) of benzyl alcohol at 0° C. The solution was stirred at room temperature for 2 hours. 10 g (78 mmol) of 4-chloropicoline was added to the above solution at room temperature, followed by stirring the solution for another 3 hours at 100° C. 200 mL of ice water was added to the above solution to yield a precipitate. The resultant precipitate was collected, washed with water, and dried to give 13.8 g (79%) of 4-benzyloxy-2-methyl-pyridine.

Compound 8 with a benzyl protected hydroxy (2-[3-(4-fluoro-benzyl)-7-benzyloxy-indolizin-1-yl]-2-oxo-N-pyridin-3-yl-acetamide) was prepared in a manner similar to that described in Example 1 by using 4-benzyloxy-2-methyl-pyridine was used as a starting material.

The protecting benzyl group was removed by using a well-known hydrogenation reduction to give compound 8.

¹H-NMR (CD₃SOCD₃) δ(ppm): 10.72 (s, 1H); 9.01(s, 1H); 8.32 (d, J=6 Hz, 1H); 8.26 (d, J=12 Hz, 2H); 7.89 (s, 1H); 7.42-7.10 (m, 6H); 7.64-7.30 (m, 6H); 4.22 (s, 2H)

ESMS calculated for (C₂₂H₁₆FN₃O₃): 389.12; found: 390.1 (M+H)⁺

### EXAMPLE 9

### Preparation of Compound 9: 2-[7-Chloro-3-(4-fluoro-benzyl)-indolizin-1-yl]-2-oxo-N-pyridin-3-yl-acetamide

Compound 9 was prepared in a manner similar to that described in Example 1.

¹H-NMR (CDCl₃, 300 MHz) δ(ppm): 9.46 (s, 1H); 8.82 (d, J=2.4 Hz, 1H); 8.68 (d, J=2.1 Hz, 1H); 8.42 (d, J=5.1 Hz, 1H); 8.31-8.23 (m, 1H); 8.09 (s, 1H); 7.74 (d, J=7.5 Hz, 1H); 7.34 (dd, J=5.1 Hz, 8.1 Hz, 1H); 7.18-7.14 (m, 2H); 7.04-6.98 (m, 2H); 6.89 (dd, J=2.4 Hz, 7.5 Hz, 1H); 4.22 (s, 2H)

ESMS calculated for (C₂₂H₁₅ClFN₃O₂): 407.82; found: 408.0 (M+H)⁺

### EXAMPLE 10

### Preparation of Compound 10: 2-[3-(4-fluoro-benzyl)-indolizin-1-yl]-2-oxo-N-(2,4-dichloro-pyridin-3-yl)-acetamide

Compound 10 was prepared in a manner similar to that described in Example 1.

¹H-NMR (CDCl₃) δ(ppm): 9.44 (s, 1H); 8.60 (d, J=12 Hz, 1H); 8.24 (d, J=10 Hz, 1H), 7.96 (s, 1H); 7.83 (d, J=12 Hz, 1H); 7.39 (d, J=10 Hz, 1H); 7.38-6.89 (m, 6H); 4.21 (s, 2H)

ESMS calculated for (C₂₂H₁₄Cl₂FN₃O₂): 441.04; found: 442.0 (M+H)⁺

### EXAMPLE 11

### Preparation of Compound 11: 2-[3-(4-cyano-benzyl)-indolizin-1-yl]-2-oxo-N-pyridin-2-yl-acetamide

Compound 11 was prepared in a manner similar to that described in Example 1.

¹H-NMR (CDCl₃) δ(ppm): 9.84 (s, 1H); 8.68 (d, J=12 Hz, 1H); 8.41-8.28 (m, 2H); 8.08 (s, 1H); 8.13 (s, 1H); 7.81-7.73 (m, 2H); 7.61 (d, J=12.5 Hz, 2H); 7.41-7.32 (m, 3H); 7.13-6.84 (m, 2H); 4.36 (s, 2H)

ESMS calculated for (C₂₃H₁₆N₄O₂): 380.13; Found: 381.2 (M+H)⁺

### EXAMPLE 12

### Preparation of Compound 12: 2-(3-benzyl-indolizin-1-yl)-2-oxo-N-pyridin-2-yl-acetamide

Compound 12 was prepared in a manner similar to that described in Example 1.

¹H NMR (CDCl₃) δ(ppm): 4.24 (s, 2H); 6.92 (t, J=7 Hz, 1H); 7.20-7.41 (m, 7H); 7.68 (m, 1H); 7.89 (d, J=7 Hz, 1H); 8.10 (s, 1H); 8.55-8.61(m, 1H); 8.65 (d, J=9 Hz, 1H); 9.60 (s, 1H)

ESMS calculated for (C₂₂H₁₇N₃O₂): 355.1; found: 356.1 (M+H)⁺

### EXAMPLE 13

### Preparation of Compound 13: 2-[3-(4-chloro-benzyl)-indolizin-1-yl]-2-oxo-N-pyridin-2-yl-acetamide

Compound 13 was prepared in a manner similar to that described in Example 1.

¹H NMR (CDCl₃) δ(ppm): 9.89 (s, 1H); 8.81 (m, 1H); 8.38 (m, 2H); 8.07 (s, 1H); 7.78 (m, 2H); 7.36 (m, 3H); 7.08 (m, 2H); 6.89 (m, 1H); 4.23 (s, 2H)

ESMS calculated (C₂₂H₁₆ClN₃O₂): 389.1; found: 390.1 (M+H)⁺

### EXAMPLE 14

### Preparation of Compound 42: 2-[3-(4-fluoro-benzyl)-indolizin-1-yl]-2-oxo-N-pyridin-2-yl-acetamide

Compound 12 was prepared in a manner similar to that described in Example 1.

¹H NMR (CDCl₃) δ(ppm): 9.77 (s, 1H); 8.66 (d, J=9.0 Hz, 1H); 8.40 (m, 2H); 8.06 (s, 1H); 7.81 (m, 3H); 7.35 (m, 1H); 7.26 (m, 4H); 4.24 (s, 2H)

ESMS calculated (C₂₂H₁₆FN₃O₂): 373.1; found: 374.1 (M+H)⁺

### EXAMPLE 15

### Preparation of Compound 15: 2-[3-(4-fluoro-benzyl)-7-hydroxy-indolizin-1-yl]-2-oxo-N-pyridin-2-yl-acetamide

Compound 15 was prepared in a manner similar to that described in Example 8.

¹H-NMR (CD₃COCD₃) δ(ppm): 9.93 (s, 1H); 8.30-8.39 (m, 2H); 8.12-8.02 (m, 2H); 7.90-7.76 (m, 2H); 7.38-7.28 (m, 2H); 7.18-7.02 (m, 3H); 6.76-7.70 (m, 1H); 4.22 (s, 2H)

ESMS calculated for (C₂₂H₁₆FN₃O₃): 389.12; found: 390.1 (M+H)⁺

### EXAMPLE 16

### Preparation of Compound 16: 2-[3-(4-cyano-benzyl)-indolizin-1-yl]-2-oxo-N-pyridin-4-yl-acetamide

Compound 16 was prepared in a manner similar to that described in Example 1.

¹H NMR (CDCl₃) δ(ppm): 4.36 (s, 2H); 6.95 (t, J=3.8 Hz, 1H); 7.3-7.5 (m, 3H); 7.6-7.7 (m, 4H); 7.80 (d, J=3.9 Hz, 1H); 8.05 (s, 1H); 8.5-8.7 (m, 3H); 9.60 (s, 1H)

ESMS calculated (C₂₃H₁₆N₄O₂): 380.13; found: 381.1 (M+H)⁺

### EXAMPLE 17

### Preparation of Compound 17: 2-[3-(4-fluoro-benzyl)-indolizin-1-yl]-2-oxo-N-pyridin-4-yl-acetamide

Compound 17 was prepared in a manner similar to that described in Example 1.

¹H NMR (DMSO-d₆) δ(ppm): 11.01 (s, 1H); 8.49 (d, J=6.3 Hz, 2H); 8.47-8.39 (m, 2H); 7.81 (d, J=6.3 Hz, 2H); 7.46-7.53 (m, 2H); 7.31-7.36 (m, 2H); 7.12-7.18 (m, 3H); 4.31 (s, 2H)

ESMS calculated (C₂₂H₁₆FN₃O₂): 373.38; found 374.4 (M+H)⁺

### EXAMPLE 18

### Preparation of Compound 18: 2-[3-(4-cyano-benzyl)-indolizin-1-yl]-2-oxo-N-(3,5-dichloro-pyridin-4-yl)-acetamide

Compound 18 was prepared in a manner similar to that described in Example 1.

¹H-NMR (CDCl₃) δ(ppm): 9.48 (s, 1H); 8.66 (d, J=12 Hz, 1H); 8.59 (s, 2H); 8.03(s 1H); 7.81 (d, J=10, 1H); 7.62 (d, J=11.5 Hz, 2H); 7.41 (m, 1H); 7.32 (d, J=11.5 Hz, 1H); 6.95 (m, 1H); 4.32 (s, 2H)

ESMS calculated for (C₂₃H₁₄Cl₂N₄O₂): 448.05; found: 449.1 (M+H)⁺

### EXAMPLE 19

### Preparation of Compound 19: 2-[3-(4-fluoro-benzyl)-7-hydroxy-indolizin-1-yl]-2-oxo-N-(3,5-dichloro-pyridin-4-yl)-acetamide

Compound 19 was prepared in a manner similar to that described in Example 8.

¹H-NMR (CD₃COCD₃) δ(ppm): 9.98 (s, 1H); 8.62 (s, 2H); 8.19 (d, J=11 Hz, 1H); 8.03 (s, 1H); 7.64 (s, 1H); 7.38-6.77 (m, 5H); 4.29 (s, 2H)

ESMS calculated for (C₂₂H₁₄Cl₂FN₃O₃): 457.04; found: 458.0 (M+H)⁺

### EXAMPLE 20

### Preparation of Compound 20: 2-[3-(4-fluoro-benzyl)-indolizin-1-yl]-2-oxo-N-(3,5-dichloro-pyridin-4-yl)-acetamide

Compound 20 was prepared in a manner similar to that described in Example 1.

¹H-NMR (CDCl₃) δ(ppm): 9.49 (s, 1H); 8.63 (d, J=12 Hz, 1H); 8.59 (s, 2H); 8.01(s, 1H); 7.84 (d, J=11 Hz, 1H); 7.43-6.92 (m, 6H); 4.21 (s, 2H)

ESMS calculated for (C₂₂H₁₄Cl₂FN₃O₂): 441.04; found: 440.0 (M−H)⁻

### EXAMPLE 21

### Preparation of Compound 21: 2-[3-(4-chloro-benzyl)-indolizin-1-yl]-N-(3,5-dichloro-pyridin-4-yl)-2-oxo-acetamide

Compound 21 was prepared in a manner similar to that described in Example 1.

¹H NMR (CDC₁₃) δ(ppm): 9.49 (s, 1H); 8.64 (m, 3H); 8.03 (s, 1H); 7.84 (d, J=9.0 Hz, 1H); 7.39 (m, 1H); 7.28 (m, 3H); 7.14 (d, J=9.0 Hz, 2H); 6.95 (m, 1H); 4.22 (s, 2H)

ESMS calculated (C₂₂H₁₆Cl₂N₃O₂): 457.0; found: 458.0 (M+H)⁺

### EXAMPLE 22

### Preparation of Compound 22: 2-[3-(4-fluoro-benzyl)-7-hydroxy-indolizin-1-yl]-2-oxo-N-pyridin-4-yl-acetamide

Compound 22 was prepared in a manner similar to that described in Example 8.

¹H-NMR (CD₃SOCD₃) δ(ppm); 10.93 (s, 1H); 10.85 (s, 1H); 8.46 (d, J=4.5 Hz, 2H); 8.23 (d, J=9 Hz, 1H); 7.82 (s, 1H), 7.78 (d, J=4.5 Hz, 2H); 7.36-7.10 (m, 5H); 4.21 (s, 2H)

ESMS calculated for (C₂₂H₁₆FN₃O₃): 389.12; found: 390.1 (M+H)³⁰

### EXAMPLE 23

### Preparation of Compound 23: 4-[1-(pyridin-4-yl-aminooxalyl)-indolizin-3-yl-methyl]-benzamide

Compound 23 was prepared in a manner similar to that described in Example 1.

¹H NMR (DMSO-d₆) δ(ppm): 4.40 (s, 2H); 7.18 (t, J=6.9 Hz, 1H); 7.30-7.56 (m, 5H); 7.80-7.86 (m, 3H); 7.90-7.96 (br, 2H); 8.38-8.52 (m, 4H); 11.00 (s, 1H)

ESMS calculated for (C₂₃H₁₈N₄O₃): 398.1; found: 399.1 (M+H)⁺

### EXAMPLE 24

### Preparation of Compound 24: 2-[3-(4-chloro-benzyl)-indolizin-1-yl]-2-oxo-N-pyridin-4-yl-acetamide

Compound 24 was prepared in a manner similar to that described in Example 1.

¹H NMR (CDCl₃) δ(ppm): 9.59 (s, 1H); 8.65 (d, J=9.0 Hz, 1H); 8.58 (d, J=6.0 Hz, 2H); 8.09 (s, 1H); 7.84 (d, J=6.0 Hz, 1H); 7.66 (m, 2H); 7.40 (m, 1H); 7.38 (m, 1H); 7.15 (d, J=6.0 Hz, 2H); 6.96 (m, 1H); 4.24 (s, 2H)

ESMS calculated for (C₂₂H₁₆ClN₃O₂): 389.1; found: 390.1 (M+H)⁺

### EXAMPLE 25

### Preparation of Compound 25: 2-(3-benzyl-indolizin-1-yl)-2-oxo-N-pyridin-4-yl-acetamide

Compound 25 was prepared in a manner similar to that described in Example 1.

¹H NMR (CDCl₃) δ(ppm): 4.20 (s, 2H); 6.80 (t, J=7 Hz, 1H); 6.95-7.40 (m, 1H); 7.12-7.30 (m, 6H); 7.68 (t, J=8 Hz, 1H); 7.78 (d, J=7 Hz, 1H); 8.02 (s, 1H); 8.26-8.34 (m, 2H); 8.60 (d, J=9 Hz, 1H); 9.82 (s, 1H)

ESMS calculated for (C₂₂H₁₇N₃O₂): 355.1; found: 356.1 (M+H)⁺

### EXAMPLE 26

### Preparation of Compound 26: 2-[3-(4-cyano-benzyl)-indolizin-1-yl]-2-oxo-N-(2,3,5-trichloro-pyridin-4-yl)-acetamide

Compound 26 was prepared in a manner similar to that described in Example 1.

¹H-NMR (CDCl₃) δ(ppm): 9.57 (s, 1H); 8.64 (d, J=12 Hz, 1H); 8.41 (s, 1H); 8.07 (s, 1H); 7.80 (d, J=12 Hz, 1H); 7.61 (d, J=12.5 Hz, 2H); 7.42 (m, 1H); 7.31 (d, J=12.5 Hz, 2H); 6.98 (m, 1H); 4.32 (s, 2H)

ESMS calculated for (C₂₃H₁₃Cl₃N₄O₂): 482.01; found: 483.1.1 (M+H)⁺

### EXAMPLE 27

### Preparation of Compound 27: 2-[7-chloro-3-(4-cyano-benzyl)-indolizin-1-yl]-2-oxo-N-pyridin-4-yl-acetamide

Compound 27 was prepared in a similar manner as described in Example 1.

¹H NMR (CDCl₃) δ(ppm): 4.32 (s, 2H); 6.92 (dd, J=7 Hz, 2 Hz, 1H); 7.20-7.35 (m, 3H); 7.55-7.72 (m, 4H); 8.09 (s, 1H); 8.59 (d, J=6 Hz, 2H); 8.68 (d, J=2 Hz, 1H); 9.52 (s, 1H)

ESMS calculated for (C₂₃H₁₅ClN₄O₂): 414.1; found: 415.1 (M+H)⁺

### EXAMPLE 28

### Preparation of Compound 28: 2-[7-chloro-3-(4-chloro-benzyl)-indolizin-1-yl]-2-oxo-N-(3,5-dichloro-pyridin-4-yl)-acetamide

Compound 28 was prepared in a manner similar to that described in Example 1.

¹H NMR (300 MHz, CDC₁₃) δ(ppm): 9.49 (s, 1H); 8.65 (d, J=2.4 Hz, 1H); 8.57 (s, 2H); 8.00 (s, 1H); 7.72 (d, J=7.2 Hz, 1H); 7.26 (d, J=8.4 Hz, 2H); 7.17 (d, J=8.4 Hz, 2H); 6.89 (dd, J=7.2 Hz, 2.4 Hz, 1H); 4.19 (s, 2H)

ESMS calculated for (C₂₂H₁₄Cl₄N₃O₂): 490.98; found: 492. 1 (M+H)⁺

### EXAMPLE 29

### Preparation of Compound 29: 2-[3-(4-methoxy-benzyl)-indolizin-1-yl]-2-oxo-N-(3,5-dichloro-pyridin-4-yl)-acetamide

Compound 29 was prepared in a manner similar to that described in Example 1.

¹H NMR (CDCl₃) δ(ppm): 9.5 (s, 1H); 8.66 (d, J=9.0 Hz, 1H); 8.58 (s, 2H); 8.01 (s, 1H); 7.89 (d, J=6.9 Hz, 1H); 7.39 (t, J=7.8 Hz, 1H); 7.12 (d, J=8.7 Hz, 2H); 6.93 (t, J=6.9 Hz, 1H); 6.84 (d, J=8.7 Hz, 2H); 4.19 (s, 2H); 3.78 (s, 3H)

ESMS calculated for (C₂₃H₁₇Cl₂N₃O₃): 453.06; found: 476.1 (M+Na)⁺

### EXAMPLE 30

### Preparation of Compound 30: 2-[7-chloro-3-(4-fluoro-benzyl)-indolizin-1-yl]-2-oxo-N-(3,5-dichloro-pyridin-4-yl)-acetamide

Compound 30 was prepared in a manner similar to that described in Example 1.

¹H-NMR (CDCl₃, 300 MHz) δ(ppm): 9.42 (s, 1H); 8.69 (d, J=2.1 Hz, 1H); 8.58 (s, 2H); 8.01 (s, 1H); 7.75 (d, J=7.2 Hz, 1H); 7.17-7.13 (m, 2H); 7.03-6.97 (m, 2H); 6.90 (dd, J=2.1 Hz, 7.2 Hz, 1H); 4.20 (s, 2H)

ESMS calculated for (C₂₂H₁₃Cl₃FN₃O₂): 476.71 found: 500.0 (M+Na)⁺

### EXAMPLE 31

### Preparation of Compound 31: 2-[7-chloro-3-(4-cyano-benzyl)-indolizin-1-yl]-2-oxo-N-(3,5-dichloro-pyridin-4-yl)-acetamide

Compound 31 was prepared in a manner similar to that described in Example 1.

¹H NMR (300 MHz, CDCl₃) δ(ppm): 9.51 (s, 1H); 8.63 (d, J=2.4 Hz, 1H); 8.55 (s, 2H); 8.00 (s, 1H); 7.71 (d, J=7.2 Hz, 1H); 7.58 (d, J=8.1 Hz, 2H); 7.30 (d, J=8.1 Hz, 2H); 6.90 (dd, J=7.2 Hz, 2.4 Hz, 1H); 4.29 (s, 2H)

ESMS calculated for (C₂₃H₁₄Cl₃N₄O₂): 483.0; found: 484.0 (M+H)⁺

### EXAMPLE 32

### Preparation of Compound 32: 4-[1-(3,5-dichloro-pyridin-4-yl-aminooxalyl)-indolizin-3-yl-methyl]-benzoic acid ethyl ester

Compound 32 was prepared in a manner similar to that described in Example 1.

¹H-NMR (CDCl₃) δ(ppm): 9.47 (s, 1H); 8.67 (d, J=8.7 Hz, 1H); 8.57 (s, 1H); 8.06 (s, 1H); 7.98 (d, J=8.1 Hz, 2H); 7.82 (d, J=7.2 Hz, 1H); 7.43-737 (m, 1H); 7.26 (s, 2H); 6.95-6.93 (m, 1H); 4.34 (q, J=7.2 Hz, 14.4 Hz, 2H); 4.31 (s, 2H), 1.37 (t, J=7.2 Hz, 3H)

ESMS calculated for (C₂₅H₁₉Cl₂N₃O₄): 495.08; found: 494.2 (M−H)⁻

### EXAMPLE 33

### Preparation of Compound 33: 4-[1-(3,5-dichloro-pyridin-4-yl-aminooxalyl)-indolizin-3-yl-methyl]-benzoic acid

Compound 33 was prepared in a manner similar to that described in Example 1.

¹H-NMR (CD₃OD) δ(ppm): 8.62-8.55 (m, 3H); 8.16 (d, J=6.0 Hz, 1H); 7.90-7.88 (m, 2H); 7.67 (s, 1H); 7.48-7.42 (m, 1H); 7.25 (s, 2H); 7.06-6.98 (m, 1H); 4.34 (s, 2H)

ESMS calculated for (C₂₃H₁₅Cl₂N₃O₄): 467.04; found: 468.0 (M+H)⁺

### EXAMPLE 34

### Preparation of Compound 34: 2-[3-(4-fluoro-benzyl)-7-methoxy-indolizin-1-yl]-2-oxo-N-(3,5-dichloro-pyridin-4-yl)-acetamide

Compound 34 was prepared in a manner similar to that described in Example 1.

¹H-NMR (CDCl₃, 300 MHz) δ(ppm): 9.55 (s, 1H); 8.57 (s, 2H); 8.09 (d, J=2.7 Hz, 1H); 7.88 (s, 1H); 7.69 (dd, J=0.3 Hz, 7.2 Hz, 1H); 7.19-7.14 (m 2H); 7.01-6.95 (m, 2H); 6.63 (dd, J=2.7 Hz, 7.2 Hz, 1H); 4.16 (s, 2H); 3.97 (s, 3H)

ESMS calculated for (C₂₃H₁₆Cl₂FN₃O₃) (M⁺): 471.60; found: 494.0 (M+Na)⁺

### EXAMPLE 35

### Preparation of Compound 35: 2-[3-(4-fluoro-benzyl)-indolizin-1-yl]-2-oxo-N-(3-chloro-pyridin-4-yl)-acetamide

Compound 35 was prepared in a manner similar to that described in Example 1.

¹H-NMR (CD₃ COCD₃) δ(ppm): 10.25 (s, 1H); 8.67-8.61 (m, 2H); 8.52 (s, 2H); 8.37 (d, J=10 Hz, 1H); 7.94 (s, 1H); 7.60-7.09 (m, 6H); 4.21 (s, 2H)

ESMS calculated for (C₂₂H₁₅ClFN₃O₂): 407.08; found: 408.0 (M+H)⁺

### EXAMPLE 36

### Preparation of Compound 36: 2-(3-cyclopropylmethyl-indolizin-1-yl)-N-(3,5-dichloro-pyridin-4-yl)-2-oxo-acetamide

Compound 36 was prepared in a manner similar to that described in Example 1.

¹H-NMR (CDCl₃) δ(ppm): 9.50 (s, 1H); 8.67-85.7 (m, 3H); 8.06-8.03 (m, 2H); 7.43-7.38 (m, 1H); 7.07-7.02 (m, 1H) 2.74 (d, J=6.6 Hz, 2H); 1.30-1.18 (m, 1H); 0.70-0.64 (m. 2H); 0.28-0.25 (m, 2H)

ESMS calculated for (C₁₉H₁₅Cl₂N₃ O₂): 387.05; Found: 410.0 (M+Na)⁺

### EXAMPLE 37

### Preparation of Compound 37: 2-(3-methyl-indolizin-1-yl)-2-oxo-N-(3,5-dichloro-pyridin-4-yl)-acetamide

Compound 37 was prepared in a manner similar to that described in Example 1.

¹H NMR (300 MHz, DMSO-D₆), δ(ppm): 8.90 (s, 2H); 8.38 (d, J=6.9 Hz, 1H); 7.85 (d, J=9.0 Hz, 1H); 7.11-7.32 (m, 4H); 2.50 (s, 3H)

ESMS calculated for (C₁₁H₁₀NO₂): 347. 1; found: 346.1 (M−H)⁻

### EXAMPLE 38

### Preparation of Compound 38: 2-[3-(4-cyano-benzyl)-indolizin-1-yl]-2-oxo-N-(3,5-dichloro-1-oxy-pyridin-4-yl)-acetamide

Compound 38 was prepared in a manner similar to that described in Example 1.

¹H NMR (300 MHz, DMSO-D₆) δ(ppm): 9.74 (br, s, 1H); 8.65 (d, J=9 Hz, 2H); 8.20 (d, J=7.2 Hz, 2H); 8.06 (s, 1H); 7.8-7.6 (m, 5H); 7.4 (m, 1H); 7.35 (d, J=7.8 Hz, 2H); 6.9 (m, 2H); 4.34 (s, 2H)

ESMS calculated for (C₂₃H₁₆N₄O₃): 396.12; found: 397.1 (M+H)⁺

### EXAMPLE 39

### Preparation of Compound 39: 2-[3-(4-fluro-benzyl)-indolizin-1-yl]-2-oxo-N-(3,5-dichloro-1-oxy-pyridin-4-yl)-acetamide

Compound 39 was prepared in a manner similar to that described in Example 1.

¹H-NMR (CD₃COCD₃) δ(ppm): 8.41 (d, J=10, 1H); 8.22 (d, J=8, 1H); 7.43-6.95 (m, 9H); 4.36 (s, 2H)

ESMS calculated for (C₂₂H₁₄FCl₂N₃O₃): 457.04; found: 457.0

### EXAMPLE 40

### Preparation of Compound 40: N-(3,5-Dichloropyridin-4-yl)-2-[3-(4-hydroxy-benzyl)-indolizin-1-yl]-2-oxo-acetamide

Compound 40 was prepared in a manner similar to that described in Example 1.

¹H NMR (DMSO-d₆): δ10.96 (s, 1H), 9.30 (s, 1H), 8.74 (s, 2H), 8.45 (d, J=9.0 Hz, 1H), 8.38 (d, J=7.2 Hz, 1H), 7.52 (t, J=8.0 Hz, 1H), 7.36 (s, 1H), 7.15 (t, J=6.9 Hz, 1H), 7.08 (d, J=8.4 Hz, 2H), 6.70 (d, J=8.4 Hz, 2H), 4.16 (s, 2H)

ESMS calculated for (C₂₂H₁₅Cl₂N₃O₃): 439.05; Found: 440.0 (M+1)⁺

### EXAMPLE 41

### Preparation of Compound 41: N-(3,5-dichloro-1-oxy-pyridin-4-yl)-2-[3-(4-hydroxy-benzyl)-indolizin-1-yl]-2-oxo-acetamide

Compound 41 was prepared in a manner similar to that described in Example 1.

¹H-NMR (DMSO-d₆) δ(ppm), 10.76(s, 1H), 9.29(s, 1H), 8.71(s, 1H), 8.46-8.37(m, 2H), 7.51(t, J=8,7 Hz, 1H), 7.36(s, 1H), 7.17-7.06(m, 3H), 6.71(d, J=6.9 Hz, 2H), 5.75(s, 1H), 4.16(s, 2H)

ESMS calculated for (C₂₂H₁₅Cl₂N₃O₄): 455.04; Found: 456.4 (M+1)⁺

### EXAMPLE 42

### Preparation of Compound 42: N-(3,5-dichloro-1-oxy-pyridin-4-yl)-2-[3-(4-methoxy-benzyl)-indolizin-1-yl]-2-oxo-acetamide

Compound 42 was prepared in a manner similar to that described in Example 1.

¹H-NMR (CDCl₃) δ(ppm), 9.34(s, 1H), 8.64(d, J=9 Hz, 1H), 8.26(s, 2H), 7.98(s, 1H), 7.92(d, J=6 Hz,1H), 7.40(t, J=9 Hz,1H), 7.14(d, J=8.4 Hz, 2H),6.94(t, J=9 Hz, 1H),6.83(d, J=7.8 Hz,2H),4.19(s,2H), 3.78(s,3H);

ESMS calculated for (C₂₃H₁₇Cl₂N₃O₄): 469.06; Found: 470.4 (M+1)⁺

### EXAMPLE 43

### Preparation of Compound 43: N-(3,5-Dichloropyridin-4-yl)-2-[7-fluoro-3-(4-fluoro-benzyl)-indolizin-1-yl]-2-oxo-acetamide

Compound 43 was prepared in a manner similar to that described in Example 1.

¹H-NMR (CDCl₃) δ(ppm): 9.45 (s, 1H), 8.58 (s, 2H), 8.35 (dd, J₁=2.7 Hz, J₂=9.6 Hz, 1H), 8.17 (d, J=7.9 Hz, 1H), 7.81 (dd, J₁=5.1 Hz, J₂=7.5 Hz, 1H), 7.14-7.18 (m, 2H), 6.90-7.00 (m, 3H), 6.77-6.83 (m, 1H), 4.21 (s, 2H)

ESMS calculated for (C₂₂H₁₃Cl₂F₂N₃O₂): 459.04; Found: 460.0 (M+H)⁺

### EXAMPLE 44

### Preparation of Compound 44: N-(3,5-Dichloro-1-oxy-pyridin-4-yl)-2-[3-(4-chloro-benzyl)-indolizin-1-yl]-2-oxo-acetamide

Compound 44 was prepared in a manner similar to that described in Example 1.

¹H-NMR (CDCl₃) δ(ppm), 9.31(s, 1H), 8.65 (d, J=12, 1H), 8.24 (s, 2H), 8.01(s, 1H), 7.84 (d, J=11, 1H ), 7.43-6.92 (m, 6H), 4.22(s, 2H)

ESMS calculated for (C₂₂H₁₄Cl₃N₃O₃): 473.01; Found: 474.1(M+H)⁺

### EXAMPLE 45

### Preparation of Compound 45: N-(3,5-Dichloro-1-oxy-pyridin-4-yl)-2-[3-(4-cyano-benzyl)-indolizin-1-yl]-2-oxo-acetamide

Compound 45 was prepared in a manner similar to that described in Example 1.

¹H-NMR (CDCl₃) δ(ppm), 9.38(s, 1H), 8.66 (d, J=12, 1H), 8.23 (s, 2H), 8.03(s, 1H), 7.81 (d, J=10, 1H ), 7.62 (d, J=11.5, 2H), 7.41-6.91(m, 6H), 4.32(s, 2H)

ESMS calculated for (C₂₃H₁₄Cl₂N₄O₃): 464.04; Found: 465.1 (M+H)⁺

### EXAMPLE 46

### Preparation of Compound 46: N-(3,5-Dichloro-1-oxy-pyridin-4-yl)-2-[7-chloro-3-(4-fluoro-benzyl)-indolizin-1-yl]-2-oxo-acetamide

Compound 46 was prepared in a manner similar to that described in Example 1.

¹H-NMR (CDCl₃, 300 MHz): δ9.21 (s, 1H), 8.68 (d, J=2.1 Hz, 1H), 8.27 (s, 2H), 7.98 (s, 1H), 7.75 (d, J=7.5 Hz, 1H), 7.17-7.12 (m, 2H), 7.03-6.97(m, 2H), 6.91 (dd, J=2.1, 7.5 Hz, 1H), 4.20 (s, 2H)

ESMS calculated for (C₂₂H₁₃Cl₃FN₃O₃):(M⁺=491.00), Found: 492.01 (M⁺+1)

### EXAMPLE 47

### Preparation of Compound 47: N-(3,5-Dichloro-1-oxy-pyridin-4-yl)-2-[7-fluoro-3-(4-fluoro-benzyl)-indolizin-1-yl]-2-oxo-acetamide

Compound 47 was prepared in a manner similar to that described in Example 1.

¹H-NMR (CDCl₃) δ(ppm): 9.31 (s, 1H), 8.32 (dd, J₁=2.4 Hz, J₂=7.8 Hz, 2H), 8.27 (s, 2H), 7.82 (dd, J₁=J₂=7.8 Hz), 7.07-7.17 (m, 3H), 6.94-7.01 (m, 3H), 6.75-6.83 (m, 1H). 4.19 (s, 2H)

ESMS calculated for (C₂₂H₁₃Cl₂F₂N₃O₃): 475.03; Found: 476.0 (M+H)⁺

### EXAMPLE 48

### Analytical Data for Compound 48 2-(3-(4-cyanobenzyl)-indolizin-1-yl)-N-(3-methyl-isothiazol-5-yl)-2-oxo-acetamide

¹H NMR (CDCl₃). 2.47 (s, 3H), 4.34 (s, 2H), 6.78 (s, 1H), 6.98 (m, 1H), 7.31 (m 3H), 7.61 (d, J=7.2, 2H), 7.80 (d, J=6.9, 1H), 7.85 (s, 1H), 8.68 (d, J=8.7, 1H), 10.36 (s, 1H).

ESMS calcd (C₂₂H₁₆N₄O₂S): 400.1; found: 399.1 (M−H)^{+.}

### EXAMPLE 49

### Analytical Data for Compound 49: 2-[3-(4-Fluoro-benzyl)-indolizin-1-yl]-N-(3-methyl-isothiazol-5-yl)-2-oxo-acetamide

¹H NMR (CDCl₃). 2.40 (s, 3H), 4.22 (s, 2 H), 6.77 (s, 1H), 6.98 (m, 3H), 7.18 (m, 2H), 7.41 (m, 1H), 7.84 (m, 1H), 8.16 (s.1H), 8.62 (m, 1H), 10.20 (s, 1H)

ESMS calcd (C₂₁H₁₆FN₃O₂S): 393; found: 394 (M+H)^{+.}

### EXAMPLE 50

### Analytical Data for Compound 50: 2-[3-benzyl-indolizin-1-yl]-N-(3-methyl-isothiazol-5-yl)-2-oxo-acetamide

¹H NMR (CDCl₃). 2.40 (s, 3H), 4.23 (s, 2 H), 6.74 (s, 1H), 6.90 (m, 1H), 7.18-7.41 (m, 6H), 7.81 (m, 1H), 8.16 (s, 1H), 8.62 (m, 1H), 10.22 (s, 1H)

ESMS calcd (C₂₁H₁₇N₃O₂S): 375; found: 376 (M+H)^{+.}

### EXAMPLE 51

### Analytical Data for Compound 51: 2-[3-(4-Chloro-benzyl)-indolizin-1-yl]-N-(3-methyl-isothiazol-5-yl)-2-oxo-acetamide

¹H NMR (CDCl₃). 4.22 (s, 2H), 6.78 (s, 1H), 6.95 (m, 1H), 7.13 (d, J=8.1 Hz, 2H), 7.26 (m, 2H), 7.41 (m, 1H), 7.84 (dd, 1H), 8.15 (s, 1H), 8.65 (dd, 1H), 10.21 (s, 1H)

ESMS calcd (C₂₁H₁₆ClN₃O₂S): 409.1; found: 410.1 (M+H)^{+.}

### EXAMPLE 52

### Analytical Data for Compound 52: 2-[3-(4-Chloro-benzyl)-indolizin-1-yl]-2-oxo-N-quinolin-6-yl-acetamide

¹H NMR (CDCl₃). 4.22 (s, 2H), 6.92 (m, 1H), 7.50 (m, 5H), 7.84 (m, 2H), 8.13 (m, 2H), 8.57 (s, 1H), 8.67 (d, J=9.0 Hz, 1H), 8.85 (s, 1H), 9.71 (s, 1H)

ESMS calcd (C₂₆H₁₈ClN₃O₂): 439.1; found: 440.1 (M+H)^{+.}

### EXAMPLE 53

### Analytical Data for Compound 53: 2-[3-(5-cyano-thiophen-2-ylmethyl)-indolizin-1-yl]-N-(3-methyl-isothiazol-5-yl)-2-oxo-acetamide)

¹H-NMR (CDCl₃): 10.40(s, 1H), 8.66(d, J=9.0 Hz, 1H), 8.24(s, 1H), 7.88(d, J=6.9 Hz, 1H), 7.49-7.42(m, 2H), 7.03(t, J=6.0 Hz, 1H), 6.88(d, J=3.9 Hz, 1H), 6.79(s, 1H), 4.48(s, 2H), 2.46(s, 3H)

ESMS clcd for C₂₀H₁₄N₄O₂S₂: 406.06; Found: 407.0 (M+H)^{+.}

### EXAMPLE 54

### Analytical Data for Compound 54: 2-[3-(3-Cyano-benzyl)-indolizin-1-yl]-N-(3-methyl-isothiazol-5-yl)-2-oxo-acetamide

¹H-NMR (CDCl₃): 10.43 (s, 1H), 8.67(d, J=9.0 Hz, 1H), 8.17(s, 1H), 7.82(d, J=6.9 Hz, 1H),7.59-7.40(m, 4H), 6.99(t, J=6.6 Hz, 1H), 6.78(s, 1H), 4.30(s, 2H), 2.46(s, 3H)

ESMS clcd for C₂₂H₁₆N₄O₂S: 400.10; Found: 401.0 (M+H)^{+.}

### EXAMPLE 55

### Analytical Data for Compound 55: 4-{2-[3-(4-Cyano-benzyl)-indolizin-1-yl]-2-oxo-acetylamino}-benzamide

¹H NMR (DMSO-d₆) 4.45 (s, 2H), 7.15 (m, 1 H), 7.55 (m, 3H), 7.8-8.0 (m, 6H), 8.40 (m, 2H), 10.82 (s, 1H)

ESMS calcd (C₂₅H₁₈N₄O₃): 422.14; found: 423.2 (M+H)^{+.}

### EXAMPLE 56

### Preparation of Compound 56: 3-[1-(3-methyl-isothiazol-5-ylaminooxalyl)-indolizin-3-ylmethyl]-benamide

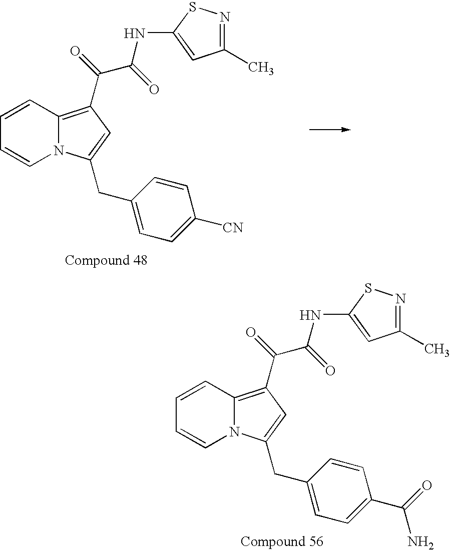

To a stirred suspension of Compound 48 (50 mg, 0.125 mmol) in DCM (2 mL) and 20% NaOH (1.5 mL) was added H₂O₂ (30%, 0.5 mL) at 0° C. After 2 h stirring at rt, EtOAc (20 mL) was added. The mixture was washed with H₂O (3×10 mL), 5% citric acid and brine, dried (Na₂SO₄). Removal of the solvent afforded the crude product as yellow solid which was washed with EtOAc/Et₂O. The product was obtained as yellow powder (30 mg, yield 57%).

¹H-NMR (DMSO-d₆) δ2.4 (s, 3H), 4.38 (s, 2H), 6.8 (bs, 2H), 7.0 (t, 1H, J=4), 7.05 (s, 1H), 7.35 (d, 2H, J=7), 7.40 (t, 1H, J=4), 7.75 (bs, 1H), 7.85 (d, 2H, J=7), 7.9 (s, 1H), 8.08 (d, 1H, J=4), 8.60 (d, 1H, J=4)

ESMS calcd for ESMS calcd for C₂₂H₁₈N₄O₃S (418.1); found: 419.1 (M+H).

### EXAMPLE 57

### Analytical Data for Compound 57: 2-[3-(4-Chloro-benzyl)-7-methyl-indolizin-1-yl]-N-(3-methyl-isothiazol-5-yl)-2-oxo-acetamide

¹H NMR (CDCl₃). 2.45 (s, 6H), 4.20 (s, 2H), 6.80 (m, 2H), 7.13 (d, 2H), 7.27 (d, 2H), 7.72 (d, 1H), 8.09 (s, 1H), 8.45 (s, 1H), 10.68 (s, 1H)

ESMS Calcd (C₂₂H₁₈ClN₃O₂S): 423.08, found 424.0 (M+H)^{+.}

### EXAMPLE 58

### Preparation of Compound 58: 2-[3-(4-Cyano-phenoxy)-indolizin-1-yl]-N-(3-methyl-isothiazol-5-yl)-2-oxo-acetamide

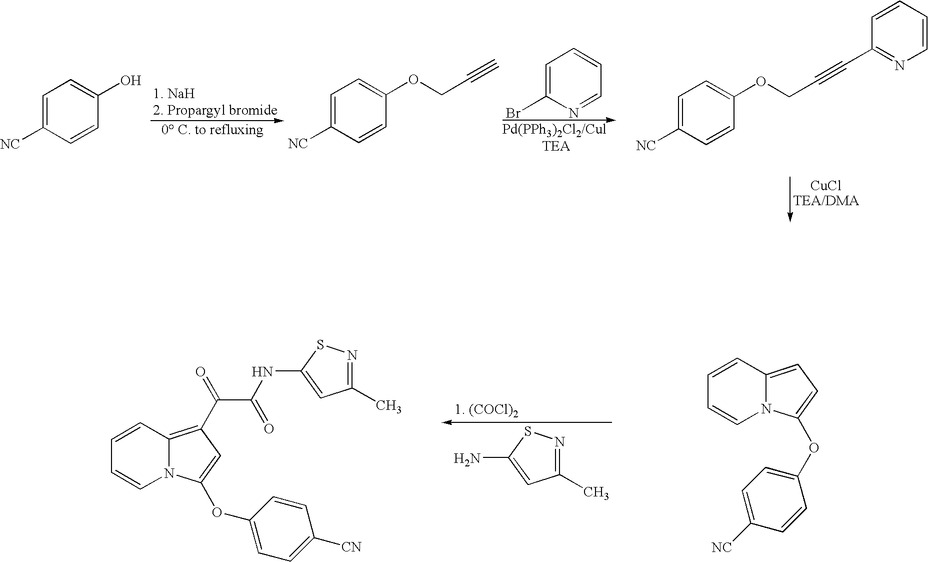

To a solution of 4-cyanophenol (1.19 g, 0.01 mol) in THF at 0° C. was added sodium hydride (0.44 g, 60% in mineral oil, 0.011 mol). 20 min later, propargyl bromide (1.23 mL, 80% in toluene, 0.011 mol) was added to the above suspension. The reaction mixture was then kept refluxing for 2 h. After purification by column chromatograph, 4-pro-2-ynyloxybenzonitrile was obtained in 82% yield.

4-Pro-2-ynyloxybenzonitrile (0.40 g, 2.59 mmol) was dissolved in TEA, followed by the successive addition of bromopyridine (0.27 mL, 2.82 mmol), Pd (pph₃)₂Cl₂ (0.04 g, 0.05 mmol), and Cul (0.01. g, 0.05 mmol). The reaction mixture was heated up to 60° C. and kept stirring at that temperature for 3 h. The reaction was cooled to room temperature, quenched with water and extracted with ethyl acetate. After purification, 4-(3-pyridin-yl-prop-2-ynyloxy)-benzonitrile was obtained (yield 76%).

The above alkynyl pyridine (0.033 g, 0.14 mmol) was added to the mixture of DMA and TEA (7:1), followed by the addition of CuCl (0.028 g, 0.28 mmol). The reaction mixture was kept stirring at 130° C. for 3 h. After workup, 4-(indolizin-3-yloxy)-benzonitrile was obtained in 85% yield.

The solution of oxalyl chloride (0.01 mL, 0.13 mmol) in anhydrous THF was cooled to 40° C., followed by the addition of 4-(indolizin-3-yloxy)-benzonitrile (0.020 g, 0.09 mmol). The resulting mixture was kept stirring at −25° C. for 40 min. To it was added 5-amino-3-methylisothiazole 0.02 g, 0.17 mmol). After workup, 2-[3-(4-Cyano-phenoxy)-indolizin-1-yl]-N-(3-methyl-isothiazol-5-yl)-2-oxo-acetamide was collected in 72% yield.

¹H NMR (CDCl₃): δ(ppm) 10.34 (s, 1H); 8.66 (d, J=9.0 Hz, 1H); 8.02 (d, J=6.0 Hz, 1H); 7.89 (s, 1H); 7.68 (d, J=9.0 Hz, 2H); 7.46 (t, J=9.0 Hz, 1H); 7.29 (d, J=9.0 Hz, 2H); 7.06 (t, J=6.0 Hz, 1H); 6.87 (s, 1H); 2.47 (s, 3H).

ESMS calcd (C₂₁H₁₄N₄O₃S): 402.1; found: 403.1 (M+H)⁺

### EXAMPLE 59

### Analytical Data for Compound 59: 2-{3-[(4-Cyano-phenyl)-methyl-amino]-indolizin-1-yl}-N-(3-methyl-isothiazol-5-yl)-2-oxo-acetamide

¹H NMR (CDCl₃). 2.56 (s, 3H), 4.73 (s, 1H), 6.81 (s, 1H), 7.15 (d, 2H), 7.27 (d, 2H), 7.45 (m, 2H), 7.65 (d, 1H), 7.83 (m, 1H), 8.06 (s, 1H), 8.19 (m, 1H), 8.65 (d, 1H), 10.30 (s, 1H)

ESMS Calcd (C₂₅H₁₈ClN₃O₂S): 459.08, found 460.0 (M+H)^{+.}

### EXAMPLE 60

### Preparation of Compound 60: 2-[3-(4-Fluoro-benzyl)-indolizin-1-yl]-N-pyridin-4-yl-acetamide

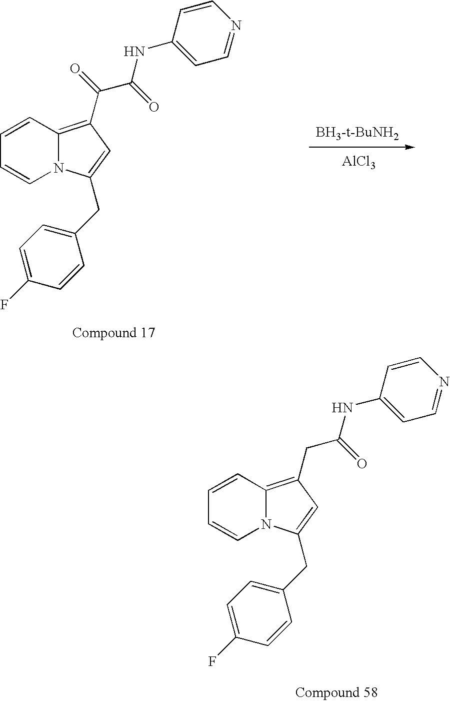

To a suspension of AlCl₃ (328 mg, 2.46 mmol) in dry THF (10 mL) cooled to 0° C. was added solid borane-tert-butylamine complex (1.07 g, 12.3 mmol) in small portions over 10 min. The resulting suspension was stirred at 0° C. for 15 min. A solution of 2-[3-(4-Fluoro-benzyl)-indolizin-1-yl]-2-oxo-N-pyridin-4-yl-acetamide (Compound 17, 918 mg, 2.46 mmol) in dry THF (5 mL) was added dropwisely to the AlC₃-t-BuNH₂ suspension. The mixture was stirred under N₂ for 18 h. Saturated sodium bicarbonate (100 mL) was added and the aqueous layer was extracted with EtOAc (2×50 mL). The EtOAc extracts were separated and washed with H₂O (3×50 mL), dried over MgSO₄ and filtered. After removal of the solvent under reduced pressure followed by silicagel chromatography (100% EtOAc) afforded the product 2-[3-(4-Fluoro-benzyl)-inidolizin-1-yl]-N-pyridin-4-yl-acetamide as a yellow solid (120 mg, 13%).

¹H-NMR (CDCl₃) 3.89 (s, 2H), 4.20 (s, 2H)), 6.52-6.57 (m, 1H), 6.72-6.77 (m, 1H), 6.97-7.04 (m, 2H), 7.11-7.16 (m, 2H), 7.31-7.34 (m, 3H), 7.35-7.66 (m, 2H), 8.42 (d, J=6.3 Hz, 1H)

ESMS calcd for C₂₂H₁₈FN₃O: 359.1; Found: 360.1 (M+H)^{+.}

### EXAMPLE 61

### Analytical Data for Compound 61: 2-[7-Chloro-3-(4-methoxy-benzyl)-indolizin-1-yl]-N-(3,5-dichloropyridin-4-yl)-2-oxo-acetamide

¹H NMR (CDCl₃): δ9.48 (s, 1H), 8.66 (d, J=2.0 Hz, 1H), 8.58 (s, 2H), 7.99 (s, 1H), 7.77 (dd, J=7.5 and 0.9 Hz, 1H), 7.08 (m, 2H), 6.86 (m, 1H), 6.84 (m, 2H), 4.16 (s, 2H), 3.77 (s, 3H)

ESMS clcd for C₂₃H₁₆Cl₃N₃O₃: 487.03; Found: 488.0 (M+H)^{+.}

### EXAMPLE 62

### Analytical Data for Compound 62: 2-[7-Chloro-3-(4-hydroxy-benzyl)-indolizin-1-yl]-N-(3,5-dichloropyridin-4-yl)-2-oxo-acetamide

¹H NMR (CDCl₃): δ11.00 (s, 1H), 9.32 (s, 1H), 8.75 (s, 2H), 8.42 (s, 1H), 8.41 (d, J=7.8 Hz, 1H), 7.36 (s, 1H), 7.24 (dd, J=7.5 and 2.1 Hz, 1H), 7.06 (d, J=8.4 Hz, 2H), 6.70 (d, J=8.4 Hz, 2H), 4.16 (s, 2H)

ESMS clcd for C₂₂H₁₄Cl₃N₃O₃: 473.01; Found: 474.0 (M+1)^{+.}

### EXAMPLE 63

### Analytical Data for Compound 63: 2-[3-(4-Hydroxy-benzyl)-indolizin-1-yl]-2-oxo-N-pyridin-3-yl-acetamide

¹H NMR (DMSO-d₆): δ10.87 (s, 1H), 9.30 (s, 1H), 8.98 (, J=2.1 Hz, 1H), 8.46 (d, J=8.7 Hz, 1H), 8.35 (m, 2H), 8.24 (d, J=8.4 Hz, 1H), 7.48 (m, 2H), 7.40 (dd, J=8.2 and 4.6 Hz, 1H), 7.13 (t, J=6.0 Hz, 1H), 7.08 (d, J=8.4 Hz, 2H), 6.70 (d, J=8.4 Hz, 2H), 4.18 (s, 2H)

ESMS clcd for C₂₂H₁₇N₃O₃: 371.13; Found: 372.1 (M+H)^{+.}

### EXAMPLE 64

### Analytical Data for Compound 64: 2-{3-[(4-Cyano-phenyl)-methyl-amino]-indolizin-1-yl}-2-oxo-N-pyridin-4-yl-acetamide

¹H NMR (CDCl₃): δ(ppm) 8.99 (s, 1H); 8.65 (m, 4H); 8.15 (s, 1H); 7.56 (m, 5H); 7.04 (m, 1H); 6.61 (d, J=9.3 Hz, 2H); 3.48 (s, 3H)

ESMS calcd (C₂₃H₁₇N₅O₂): 395.2; found: 396.2 (M+H)⁺

### EXAMPLE 65

### In vitro Assay (Inhibition of Human TNFα)

Reagents. Lipopolysaccharide (LPS, Serratia marscencens) was obtained from Sigma (St. Louis, Mo). RPMI-1640 medium and fetal calf serum (FCS) were purchased from the ATCC (Manassas, Va).

Human In Vitro Assay. Human peripheral blood cells (PBMC) were isolated by centrifugation using Ficoll-Paque (Pharmacia Biotech, Uppsala, Sweden) and suspended in RPMI-1640 medium supplemented with 10% FCS, 100 U/mL penicillin, and 100 μg/mL streptomycin. The cells were then plated in the wells of a 96-well plate at a concentration of 5×105 cells/well, and stimulated by adding LPS (1 μg/mL). Each test compound was dissolved in DMSO and added to the wells. The final DMSO concentration was adjusted to 0.25% in all cultures, including the compound-free control, and the concentrations of each test compound ranged from 0 to 10 μM. Cell-free supernatants were taken 18 h later for measurement of cytokines. Cell viability was assessed using the bioreduction of MTS [3-(4,5-dimethylthiazol-2-yl)-5-(3-carboxymethoxyphenyl)-2-(4-sulophenyl)-2H-tetrazolium] after 18 h and 48 h. Cell survival was estimated by determining the ratio of the absorbance in each of the compound-treated cultures to that in the compound-free control.

The supernatant was assayed for the amount of TNFα by using an ELISA assay with anti-human TNFα antibodies (Cell Sciences, Norwood, Mass.). The assay was carried out following the manufacturer's instructions.

Compounds 1-8, 11-32, 34, 36-38 and 48-64 were tested. Unexpectedly, 48 of the compounds tested showed IC₅₀ values lower than 10 μM, and 6 showed IC₅₀ values of 50 nM or lower. Even at the highest concentration (10 μM), none of the test compounds affected cell viability after 48 h. The most potent compounds (Compounds 2 and 48) showed IC₅₀ values of about 0.1 nM.

### EXAMPLE 66

### In vitro Assay (Inhibition of PDE4)

PDE4 was prepared from U937 human monocytic cells according to the method of Tenor et al. (Clin Exp Allegy (1995) 25:625-633). Briefly, U937 cells were homogenized in a mixture of pH 7.4 containing 10 mM Hepes, 1 mM b-mercaptoethanol, 1 mM MgCl₂, 1 mM EGTA, 137 mM NaCl, 2.7 mM KCl, 1.5 mM KH₂PO₄, 8.1 mM Na₂HPO₄, 5 μM pepstain A, 10 μM leupeptin, 50 μM PMSF, 10 μM soybean trypsin inhibitor, and 2 mM benzamindine. The homogenate was centrifuged at 200,000×g for 30 min. PDE4 activity in the supernatant was assayed in a 200 μl reaction containing 40 mM Tris-HCl, pH 7.5, 23 nM [³H]-adenosine 3′,5′ cyclic monophosphate (cAMP), 8.3 mM MgCl₂, 1.7 mM EGTA, 0.25% DMSO, and a testing compound. The assay mixture was incubated at 37° C. for 30 min and the reaction was terminated by the addition of 100 μl of yttrium silicate SPA beads (Amersham Pharmacia Biotech, Piscataway, N.J.) suspended in 18 mM ZnSO₄. The assay mixture was rotated for 3 min to ensure the binding of [³H]-5′ adenosine monophosphate to the beads. Finally, the beads was spun down, washed twice with 6 mM ZnSO₄, resuspended in 100 μl of 0.1 N NaOH, and then counted for radioactivity in a liquid scintillation counter.

Compounds 1, 2-4, 8-10, 11, 15-23, 28-32, 35, 36, and 39 were tested. All tested compounds showed IC₅₀ values lower than 5 μM, and 4 of them showed IC₅₀ values lower than 100 nM.

### EXAMPLE 67

### In vivo Assay (Edema)

Male Sprague-Dawley rats (Charles River Laboratories, Wilmington, Mass.), weighing 120-180 gram, were used throughout this study. A 1% (wt/vol) solution of lambda carrageenan (Sigma, St. Louis, Mo.) in saline was prepared freshly for each experiment. Compound 16 was formulated in 10% DMSO and 18% cremophore for intravenous administration and formulated in 1% methylcellulose (MC) (mol. Wt. 5000) for oral administration. Groups of 5 male rats were selected for study. Before carrageenan injection, compound 16 was intravenously or orally administered. Thirty minutes later, the rats were lightly anesthetized and 0.1 mL of carrageenan solution was injected by a subplantar route into the right hind paw. Paw volumes before and after carrageenan challenge were measured using hydroplethysmograph (Socrel, Varese, Italy), and the increase in volume caused by the irritant was determined after subtracting the volume of the paw before injection. Up to 69% inhibition of paw volume increase was achieved.

### Example 68

### In vivo Assay (Septic Shock)

Septic shock was elicited by two consecutive injection of E.Coli 055: B5 LPS in 9 week old female Balb/c mice (Taconic Farms, Germantown, N.Y.). The test compounds were formulated in 10% DMSO and 18% cremophore. Groups of 5 female mice weighing 19-20 gram were selected for study. E.Coli 055:B5 was reconstituted in phosphate buffered saline (PBS).

The priming injection was given in the footpad with 1.5 μg LPS per mouse. 24 h later the test compounds were intravenously or orally administered, followed by a challenge of 250 μg of LPS injected intravenously. Mortality was monitored after 24, 48 and 72 hours.

Compounds 11, 16, 17, 19, and 20 were tested. Mice in the vehicle control group were all dead after 24 hours. Mice treated with the tested compounds showed a higher survival rate. Indeed, all mice in groups treated with compounds 16 and 20 survived after 72 hours.

### EXAMPLE 69

### In vivo Assay (Crohn's Disease)

Wistar derived male or female rats (Charles River Laboratories, Wilmington, Mass.) weighing 200±20 g and fasted for 24 hours, were used. Distal colitis was induced by intra-colonic instillation of 2,4-dinitrobenzene sulfonic acid (DNBS, 25 mg in 0.5 mL 30% ethanol solution) after which 2 mL of air was gently injected through the cannula to ensure that the solution remained in the colon. Compound 20 was administered orally 24 hours and 2 hours daily for 5 days before the DNBS instillation. One control group was treated with vehicle alone, while the other was treated with both DNBS and vehicle. The animals were sacrificed 24 hours after the final administration and the colons were removed and weighed. The colon-to-body weight ratio was then calculated for each animal. The increase in ratio of DNBS+vehicle control group relative to vehicle control group was used as a base for comparison. In the treated group, a 45% deduction in the inflammatory response was observed.

### OTHER EMBODIMENTS

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present invention, and without departing from the spirit and scope thereof, can make various changes and modifications of the invention to adapt it to various usages and conditions. Thus, other embodiments are also within the scope of the following claims.

What is claimed is:

1. A compound of Formula (I): 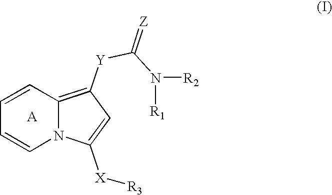
wherein Ring A is substituted or unsubstituted and is optionally fused to an aryl group;
Y is —C(R₄R₅)—, —N(R₄)—, —O—, —S—, —S(O)—, —S(O)₂—, —C(═O)—, —C(═S)—, —C(═O)—N(R₄)—, —C(═N—OR₁₂)—, —C(═N—R₁₂)—, or —N(R₄)—C(═O)—;
Z is ═O, ═S, ═N—OR₁₂ or ═NR₁₂;
R₁ and R₂ are independently —H, an unsubstituted aliphatic group, a substituted aliphatic group, an unsubstituted non-aromatic heterocylic group, a substituted non-aromatic heterocylic group, an unsubstituted aryl group or a substituted aryl group, provided that R₁ and R₂ are not both —H; or alternatively, NR₁R₂, taken together, is a substituted or unsubstituted non-aromatic nitrogen-containing heterocyclic group or a substituted or unsubstituted nitrogen-containing heteroaryl group;
R₃ is a substituted or unsubstituted aryl group or a substituted or unsubstituted aliphatic group;
X is a covalent bond, —C(R₄R₅)—, —N(R₄)—, —O—, —S—, —S(O)—, —S(O)₂—, —C(═O)—, —C(═O)—N(R₄)—, or —N(R₄)—C(═O)—;
each R₄ and R₅ is independently —H or a substituted or unsubstituted aliphatic group; and
R₁₂ is —H or a substituted or unsubstituted alkyl group or a pharmaceutically acceptable salt or prodrug thereof.

2. The compound of claim 1, wherein Ring A is optionally substituted with halo, —C₁-C₄ alkyl, —C₁-C₄ alkoxy, —C₁-C₄ haloalkyl, C₁-C₄ haloalkoxy, —C₁-C₄ acyl, amido, substituted amido, —NO₂, —CN, —OH, —NH₂ and substituted amino; Y is —C(R₄R₅) or C═O; Z is ═O; R₁ is —H; R₂ is a substituted or unsubstituted alkyl group or a substituted or unsubstituted aryl group; R₃ is a substituted or unsubstituted aryl group; and X is —C(R₄R₅)—, —N(R₄)—, —C(═O)— or —O—.

3. The compound of claim 2, wherein Y is C═O; R₂ is an unsubstituted aryl group or an aryl group substituted with lower alkyl, amido, cyano, or halo; R₃ is a substituted or unsubstituted phenyl, a substituted or unsubstituted pyridyl or a substituted or unsubstituted thienyl; and X is —CH₂—, —CH(lower alkyl)-, —NH—, —N(lower alkyl)-, —C(═O)— or —O—.

4. The compound of claim 1 of Formula (Ia): 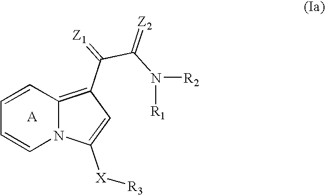
wherein Ring A is substituted or unsubstituted and is optionally fused to an aryl group;
Z₁ and Z₂ are independently ═O, ═S, ═N—OR₁₂ or ═NR₁₂;
R₁ and R₂ are independently —H, an unsubstituted aliphatic group, a substituted aliphatic group, an unsubstituted non-aromatic heterocylic group, a substituted non-aromatic heterocylic group, an unsubstituted aryl group or a substituted aryl group, provided that R₁ and R₂ are not both —H; or alternatively, NR₁R₂, taken together, is a substituted or unsubstituted non-aromatic nitrogen-containing heterocyclic group or a substituted or unsubstituted nitrogen-containing heteroaryl group R₃ is a substituted or unsubstituted aryl group or a substituted or unsubstituted aliphatic group;
X is a covalent bond, —C(R₄R₅)—, —N(R₄)—, —O—, —S—, —S(O)—, —S(O)₂—, —C(═O)—, —C(═O)—N(R₄)—, or —N(R₄)—C(═O)—;
each R₄ and R₅ are independently —H or a substituted or unsubstituted aliphatic group;
R₁₂ is —H or a substituted or unsubstituted alkyl group; or a pharmaceutically acceptable salt or prodrug thereof.

5. The compound of claim 4, wherein Ring A is optionally substituted with lower alkyl, Z₁ and Z₂ are each ═O; R₁ is —H; R₂ is a substituted or unsubstituted aryl group or a substituted or unsubsituted non-aromatic heterocylic group; R₃ is a substituted or unsubstituted aryl group; and X is —C(R₄R₅)—, —N(R₄)—, —C(═O)— or —O—.

6. The compound of claim 5, R₂ is an unsubstituted aryl group or an aryl group substituted with lower alkyl, amido, cyano, or halo; R₃ is a substituted or unsubstituted phenyl, a substituted or unsubstituted pyridyl or a substituted or unsubstituted thienyl; and X is —CH₂—, —CH(lower alkyl)-, —NH—, —N(lower alkyl)-, —C(═O)— or —O—.

7. A compound of claim 1 of Formula (Ib): 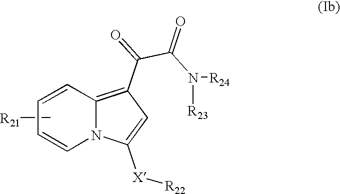
wherein:
R₂₁ occurs at each unfixed position of the ring system and each R₂₁ is independently H, lower alkyl, lower alkoxy, OH, F, Cl, Br, I, NO₂, or CN;
R₂₂ is alkyl optionally substituted with lower alkoxy, OH, CN, F, Cl, Br, I, NO₂, NH₂, C(O)NH₂, CO₂H, or CO₂R′; or aryl optionally substituted with lower alkyl, lower alkoxy, OH, CN, F, Cl, Br, I, NO₂, NH₂, or C(O)NH₂, CO₂H, or CO₂R′;
R₂₃ is H or lower alkyl;
R₂₄ is N-oxy pyridyl or pyridyl optionally substituted with F, Cl, Br, or I;
X′ is C(R′R″), N(R′), O, S, S(O), S(O)₂, C(O), C(O)—N(R′), N(R′)—C(O), or deleted;
each of R′ and R″, independently, is H, or alkyl optionally substituted with lower alkoxy, OH, CN, F, Cl, Br, I, NO₂, NH₂, or C(O)NH₂;
or a pharmaceutically acceptable salt or prodrug thereof.

8. The compound of claim 7, wherein each R₂₁ is independently H, OH, F, or Cl; R₂₂ is optionally substituted aryl; and R₂₃ is H; and X′ is CH₂.

9. The compound of claim 8, wherein R₂₂ is phenyl optionally p-substituted with lower alkoxy, OH, CN, F, Cl, Br, I, NO₂, NH₂, C(O)NH₂, CO₂H, or CO₂R′.

10. A compound selected from Compound 1-Compound 64.

11. A pharmaceutical composition comprising at least one compound according to any one of claims 1-10 and a pharmaceutically acceptable carrier.

12. The pharmaceutical composition of claim 11, further comprising one or more additional therapeutic agents.

13. The pharmaceutical composition of claim 12, wherein the additional therapeutic agent is an agent against cancer agent, an autoimmune disease, an inflammatory disorder or pain.

14. A method for treating cancer, an inflammatory disorder or an autoimmune disease comprising the step of administering to a subject in need thereof an effective amount of the pharmaceutical composition according to any one of claims 11-13.

15. A method for preventing cancer, an inflammatory disorder or an autoimmune disease comprising the step of administering to a subject in need thereof an effective amount of the pharmaceutical composition according to any one of claims 11-13.

16. A method for preventing or treating a disorder involving PDE4 or elevated levels of cytokines comprising the step of administering to a subject in need thereof an effective amount of the pharmaceutical composition according to any one of claims 11-13.

17. The method according to claim 16, wherein the disorder is characterized, mediated or exacerbated by overproduction or activity of TNFα.

18. The method according to claim 16, wherein the disorder is characterized, mediated or exacerbated by overproduction or activity of PDE4.

19. A method of inhibiting TNFα or PDE4 in a cell comprising the step of contacting the cell with an effective amount of a compound according to any one of claims 1-10.

20. A method for reducing TNFα levels in a subject comprising administering to the subject an effective amount of a compound according to any one of claims 1-10.

21. A method for suppressing inflammatory cell activation comprising the step of contacting the cell with an effective amount of a compound according to any one of claims 1-10.

22. The use of a compound according to claim 1 for the manufacture of a medicament for the prevention or treatment of cancer, an inflammatory disorder, an autoimmune disease or other condition involving PDE4 or elevated levels of cytokines, wherein the medicament comprises an effective amount of the compound.
